SEQUENCE LISTING (46 sequences)

SEQ ID NO: 1 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 2 - protein, 385 aa, Homo sapiens
MPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 3 - protein, 357 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFRLVFRGMSISRPNAVVGRCRMIRHSRDKKNEPNPQRFDRIAHTKETMLSDGLNSLTYQVLDVQRYPLYTQITVDIGTPS

SEQ ID NO: 4 - protein, 225 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQYEKIRRLLW

SEQ ID NO: 5 - protein, 24 aa, Homo sapiens
MRLREPLLSGSAAMPGASLQRACR

SEQ ID NO: 6 - protein, 20 aa, Homo sapiens
LLVAVCALHLGVTLVYYLAG

SEQ ID NO: 7 - protein, 354 aa, Homo sapiens
RDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 8 - protein, 290 aa, Homo sapiens
GPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 9 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 10 - protein, 175 aa, Homo sapiens
MNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 11 - protein, 9 aa, Homo sapiens
MIHTNLKKK

SEQ ID NO: 12 - protein, 17 aa, Homo sapiens
FSCCVLVFLLFAVICVW

SEQ ID NO: 13 - protein, 380 aa, Homo sapiens
KEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 14 - protein, 320 aa, Homo sapiens
AKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 15 - protein, 339 aa, Homo sapiens
GSSPLLDMLEHHHHHHHHMAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 16 - protein, 26 aa, Artificial Sequence
  Description of Artificial Sequence Synthetic peptide
KRRWKKNFIAVSAANRFKKISSSGAL

SEQ ID NO: 17 - protein, 220 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 18 - protein, 410 aa, Artificial Sequence
  Description of Artificial Sequence Synthetic polypeptide
MGSSHHHHHHSSGLVPRGSHMGSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGR

SEQ ID NO: 19 - protein, 9 aa, Artificial Sequence
  Description of Artificial Sequence Synthetic peptide
YPYDVPDYA

SEQ ID NO: 20 - protein, 10 aa, Artificial Sequence
  Description of Artificial Sequence Synthetic peptide
EQKLISEEDL

SEQ ID NO: 21 - protein, 38 aa, Artificial Sequence
  Description of Artificial Sequence Synthetic polypeptide
MDEKTTGWRGGHVVEGLAGELEQLRARLEHHPQGQREP

SEQ ID NO: 22 - protein, 308 aa, Artificial Sequence
  Description of Artificial Sequence Synthetic polypeptide
MADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKGSMGWDLTVKMLAGNEFQVSLSSSMSVSELKAQITQKIGVHAFQQRLAVHPSGVALQDRVPLASQGLGPGSTVLLVVDKCDEPLNILVRNNKGRSSTYEVRLTQTVAHLKQQVSGLEGVQDDLFWLTFEGKPLEDQLPLGEYGLKPLSTVFMNLRLRGG

SEQ ID NO: 23 - protein, 12 aa, Artificial Sequence
  Description of Artificial Sequence Synthetic peptide
PDRVRAVSHWSS

SEQ ID NO: 24 - protein, 8 aa, Artificial Sequence
  Description of Artificial Sequence Synthetic peptide
WSHPQFEK

SEQ ID NO: 25 - protein, 8 aa, Artificial Sequence
  Description of Artificial Sequence Synthetic peptide
DYKDDDDK

SEQ ID NO: 26 - protein, 4 aa, Artificial Sequence
  Description of Artificial Sequence Synthetic 4xHis tag
HHHH

SEQ ID NO: 27 - protein, 5 aa, Artificial Sequence
  Description of Artificial Sequence Synthetic 5xHis tag
HHHHH

SEQ ID NO: 28 - protein, 6 aa, Artificial Sequence
  Description of Artificial Sequence Synthetic 6xHis tag
HHHHHH

SEQ ID NO: 29 - protein, 7 aa, Artificial Sequence
  Description of Artificial Sequence Synthetic 7xHis tag
HHHHHHH

SEQ ID NO: 30 - protein, 8 aa, Artificial Sequence
  Description of Artificial Sequence Synthetic 8xHis tag
HHHHHHHH

SEQ ID NO: 31 - protein, 9 aa, Artificial Sequence
  Description of Artificial Sequence Synthetic 9xHis tag
HHHHHHHHH

SEQ ID NO: 32 - protein, 10 aa, Artificial Sequence
  Description of Artificial Sequence Synthetic 10xHis tag
HHHHHHHHHH

SEQ ID NO: 33 - protein, 3 aa, Artificial Sequence
  Description of Artificial Sequence Synthetic peptide
PRD

SEQ ID NO: 34 - protein, 3 aa, Artificial Sequence
  Description of Artificial Sequence Synthetic peptide
PGG

SEQ ID NO: 35 - protein, 293 aa, Artificial Sequence
  Description of Artificial Sequence Synthetic polypeptide
GPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPSPRD

SEQ ID NO: 36 - protein, 293 aa, Artificial Sequence
  Description of Artificial Sequence Synthetic polypeptide
GPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPSPGG

SEQ ID NO: 37 - protein, 302 aa, Artificial Sequence
  Description of Artificial Sequence Synthetic polypeptide
MGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPSPGGHHHHHHHH

SEQ ID NO: 38 - protein, 301 aa, Artificial Sequence
  Description of Artificial Sequence Synthetic polypeptide
MGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPSPRDHHHHHHH

SEQ ID NO: 39 - protein, 294 aa, Artificial Sequence
  Description of Artificial Sequence Synthetic polypeptide
MGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPSPGG

SEQ ID NO: 40 - protein, 9 aa, Artificial Sequence
  Description of Artificial Sequence Synthetic peptide
EEQYNSTYR

SEQ ID NO: 41 - protein, 9 aa, Artificial Sequence
  Description of Artificial Sequence Synthetic peptide
EEQFNSTFR

SEQ ID NO: 42 - protein, 9 aa, Artificial Sequence
  Description of Artificial Sequence Synthetic peptide
EEQYNSTFR

SEQ ID NO: 43 - protein, 9 aa, Artificial Sequence
  Description of Artificial Sequence Synthetic peptide
EEQFNSTYR

SEQ ID NO: 44 - protein, 20 aa, Artificial Sequence
  Description of Artificial Sequence Synthetic peptide
  SITE (1)..(20): This sequence may encompass 3-20 residues
HHHHHHHHHHHHHHHHHHHH

SEQ ID NO: 45 - protein, 8 aa, Artificial Sequence
  Description of Artificial Sequence Synthetic peptide
  SITE (1)..(8): This sequence may encompass 7-8 residues
HHHHHHHH

SEQ ID NO: 46 - protein, 308 aa, Artificial Sequence
  Description of Artificial Sequence Synthetic polypeptide
GSSPLLDMGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPSPRDHHHHHHH

(19) United States
(12) Patent Application Publication
(10) Pub. No.: US 2023/0374062 A1
(43) Pub. Date: Nov. 23, 2023

(54) METHODS OF MAKING HYPER-SIALYLATED IMMUNOGLOBULIN

(71) Applicant: Momenta Pharmaceuticals, Inc., Titusville, NJ (US)

(72) Inventors: Naveen Bhatnagar, Framingham, MA (US); James Meador, III, Framingham, MA (US)

(21) Appl. No.: 17/909,282

(22) PCT Filed: Mar. 4, 2021
(86) PCT No.: PCT/US2021/020898

§ 371 Date: Sep. 2, 2022

Related U.S. Application Data
(60) Provisional application No. 63/026,805, filed on May 19, 2020.
(60) Provisional application No. 62/985,467, filed on Mar. 5, 2020.

(51) Int. Cl. C07K 1/04; C12N 9/10

(52) U.S. Cl. CPC C07K 1/042; C12N 9/1051; C07K 2317/41; C12Y 204/01038

(57) ABSTRACT

Disclosed herein are methods galatosylating IgG antibodies, methods of preparing hypersialylated (hsIgG), e.g., using immobilized β1,4-Galactosyltransferase I (β4GalT1), as well as polypeptides comprising β1,4-Galactosyltransferase I (β4GalT1) bound to a solid support and compositions comprising the same.

### CLAIM OF PRIORITY

This application is a U.S. National Stage Application of International Application No. PCT/US2021/020898, filed on Mar. 4, 2021, which claims the benefit of U.S. Provisional Application Ser. No. 62/985,467, filed on Mar. 5, 2020, and U.S. Provisional Application Ser. No. 63/026,805, filed on May 19, 2020. The entire contents of the foregoing applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Apr. 30, 2021, is named 14131-0226WO1_SL.txt and is 62,850 bytes in size.

### TECHNICAL FIELD

The present disclosure relates to methods galatosylating IgG antibodies, methods of preparing hypersialylated (hsIgG), e.g., using immobilized β1,4-Galactosyltransferase I (β4GalT1), as well as polypeptides comprising β1,4-Galactosyltransferase I (β4GalT1) bound to a solid support and compositions comprising the same.

### BACKGROUND

Intravenous immunoglobulin (IVIg), which is prepared from the pooled plasma of human donors (e.g., pooled plasma from at least 1,000 donors), is used to treat a variety of inflammatory disorders. However, IVIg preparations have distinct limitations, such as variable efficacy, clinical risks, high costs, and finite supply. Different IVIg preparations are frequently treated as interchangeable products clinically, but it is well-known that significant differences in product preparations exist that may impact tolerability and activity in selected clinical applications. At the current maximal dosing regimens, only partial and unsustained responses are obtained in many instances. In addition, the long infusion times (4-6 h) associated with the high volume of IVIg treatment consume significant resources at infusion centers and negatively affect patient-reported outcomes, such as convenience and quality of life.

The identification of the important anti-inflammatory role of Fc domain sialylation has presented an opportunity to develop more potent immunoglobulin therapies. Commercially available IVIg preparations generally exhibit low levels of sialylation on the Fc domain of the antibodies present. Specifically, they exhibit low levels of di-sialylation of the branched glycans on the Fc region.

Washburn et al. (Proceedings of the National Academy of Sciences, USA 112: E1297-E1306 (2015)) describes a controlled sialylation process to generate highly tetra-Fc-sialylated IVIg and showed that the process yields a product with consistent enhanced anti-inflammatory activity.

### SUMMARY

Described herein are methods of galatosylating IgG antibodies comprising: (a) providing a mixture of IgG antibodies; and (b) incubating the mixture of IgG antibodies in a reaction mixture comprising: a polypeptide comprising an enzymatically active portion of human β1,4-Galactosyltransferase I (β4GalT1) bound to a solid support; and UDP-Gal, thereby producing galactosylated IgG antibodies.

Also described herein are methods of preparing hypersialylated (hsIgG) comprising: (a) providing galactosylated IgG antibodies produced as described herein; and (b) incubating the galactosylated IgG antibodies in a reaction mixture comprising: a polypeptide comprising human ST6Gal1 or enzymatically active portion thereof; and CMP-NANA, thereby producing hsIgG.

In some embodiments, the method of preparing hsIgG further comprises (c) isolating the polypeptide comprising an enzymatically active portion of human β1,4-Galactosyltransferase I (β4GalT1) bound to a solid support from the reaction mixture, thereby producing recycled β4GalT1; and repeating steps (a)-(b), wherein the β4GalT1 in the reaction mixture is the β4GalT1 isolated in step (c).

Also described herein are methods of preparing hypersialylated (hsIgG) comprising (a) providing a mixture of IgG antibodies, (b) incubating the mixture of IgG antibodies in a reaction mixture comprising: a polypeptide comprising an enzymatically active portion of human β1,4-Galactosyltransferase I (β4GalT1) bound to a solid support; and UDP-Gal, thereby producing galactosylated IgG antibodies; and (c) incubating the galactosylated IgG antibodies in a reaction mixture comprising: a polypeptide comprising human ST6Gal1 or enzymatically active portion thereof; and CMP-NANA, thereby producing hsIgG.

In some embodiments, the method of preparing hsIgG further comprises (d) isolating the polypeptide comprising an enzymatically active portion of human β1,4-Galactosyltransferase I (β4GalT1) bound to a solid support from the reaction mixture, thereby producing recycled β4GalT1; and repeating steps (a)-(c), wherein the β4GalT1 in the reaction mixture is the β4GalT1 isolated in step (d).

In some embodiments, the human β1,4-Galactosyltransferase I (β4GalT1) bound to a solid support is separated from the galactosylated IgG antibodies after step (b).

In some embodiments, the enzymatically active portion of human β4GalT1 comprises SEQ ID NO:8. In some embodiments, the polypeptide comprising the enzymatically active portion of human β4GalT1 is at least 85% identical SEQ ID NO: 37, 38, or 39, or a variant thereof having 1, 2, 3, 4, 5, 6, 7, 8, 9, or 10 amino acid substitutions, additions, or subtractions.

In some embodiments, the human ST6Gal1 or enzymatically active portion thereof comprises SEQ ID NO:14.

In some embodiments, the polypeptide comprising an enzymatically active portion of human β4GalT1 further comprises an affinity tag, wherein the affinity tag is attached to the solid support.

In some embodiments, the affinity tag is C-terminal.

In some embodiments, the at least one tag is selected from the group comprising polyhistidine, chitin binding protein (CBP), glutathione S-transferase (GST), maltose-binding protein (MBP), hemagglutinin (HA), Myc, streptavidin-binding peptide (SBP), calmodulin-tag, Spot-tag, a streptavidin tag, FLAG-tag, biotin, and combinations thereof.

In some embodiments, the polyhistidine tag comprises 3, 4, 5, 6, 7, 8, 9, 10, 11, 12, 13, 14, 15, 16, 17, 18, 19, or 20 histidines (SEQ ID NO: 44).

In some embodiments, the polyhistidine tag comprises 7 or 8 histidines (SEQ ID NO: 45).

In some embodiments, the solid support is a magnetic bead.

In some embodiments, the IgG antibodies comprise IgG antibodies isolated from at least 1000 donors.

In some embodiments, at least 70% w/w of the IgG antibodies are IgG1 antibodies.

In some embodiments, at least 90% of the donor subjects have been exposed to a virus.

In some embodiments, about 60%, 65%, 70%, 75%, 80%, or 85% of the branched glycans on the IgG antibodies in the hsIgG preparation have a sialic acid on both the α1,3 branch and the α1,6 branch.

In some embodiments, at least 60%, 65%, 70%, 75%, 80%, or 85% of the branched glycans on the Fab domain of the IgG antibodies in the hsIgG preparation have a sialic acid on both the α 1,3 arm and the α 1,6 arm that is connected through a NeuAc-α 2,6-Gal terminal linkage; and at least 60%, 65%, 70%, 75%, 80%, or 85% of the branched glycans on the Fc domain of the IgG antibodies in the hsIgG preparation have a sialic acid on both the α 1,3 arm and the α 1,6 arm that is connected through a NeuAc-α 2,6-Gal terminal linkage.

Also described herein is a polypeptide comprising: an enzymatically active portion of human β1,4-Galactosyltransferase I (β4GalT1); and an affinity tag, wherein the polypeptide is bound to a solid support.

In some embodiments, the enzymatically active portion of β4GalT1 comprises SEQ ID NO:8.

In some embodiments, the affinity tag comprises a poly-histidine tag selected from the group consisting of HHHH (SEQ ID NO:26), HHHHH (SEQ ID NO:27), HHHHHH, (SEQ ID NO:28), HHHHHHH (SEQ ID NO:29), HHHHHHHH (SEQ ID NO:30), HHHHHHHHH (SEQ ID NO:31), and HHHHHHHHHH (SEQ ID NO:32).

In some embodiments, the solid support is an agarose magnetic bead.

Also described herein is a composition comprising:
a polypeptide described herein, e.g., a polypeptide comprising an enzymatically active portion of human β1,4-Galactosyltransferase I (β4GalT1); and an affinity tag, wherein the polypeptide is bound to a solid support herein; a ST6Gal1;
UDP-Gal; CMP-NANA; and IgG antibodies.

Also described herein are methods for preparing immunoglobulin G (IgG) having a very high level of Fc sialylation. The methods described herein can provide hypersialylated IgG (hsIgG) in which greater than 70% of the branched glycans on the Fc domain are sialylated on both branches (i.e., on the alpha 1,3 branch and on the alpha 1,6 branch). HsIgG contains a diverse mixture of IgG antibody subtypes with IgG1 antibodies being the most prevalent, followed by IgG2. The diversity of the antibodies is high. The immunoglobulins used to prepare hsIgG can be obtained, for example from pooled human plasma (e.g., pooled plasma from at least 1,000-30,000 donors). The immunoglobulins can be obtained from IVIg, including commercially available IVIg. HsIgG has far higher level of sialic acid on the branched glycans on the Fc region than does IVIg. This results in a composition that differs from IVIg in both structure and activity. HsIgG can be prepared as described in WO2014/179601 or Washburn et al. (Proceedings of the National Academy of Sciences, USA 112: E1297-E1306 (2015)), both of which are hereby incorporated by reference.

Described herein are improved methods for preparing hsIgG, e.g., by immobilizing enzyme. In some embodiments, described herein, inter alia, is a method of preparing hypersialylated (hsIgG), the method comprising: (a) providing a mixture of IgG antibodies, (b) incubating the mixture of IgG antibodies in a reaction mixture comprising β1,4-Galactosyltransferase I (β4GalT1, also called B4GalT or B4Gal) bound to a solid support and UDP-Gal to produce galactosylated IgG antibodies; (c) incubating the galactosylated IgG antibodies in a reaction mixture comprising ST6Gal1 (also called ST6) and CMP-NANA, thereby creating the hsIgG preparation.

Benefits of immobilizing enzyme include, e.g., ability to glycosylate multiple hs-IVIG batches using the same enzyme, and simplifying enzyme separation from the hs-IVIG product.

In some embodiments, the β4GalT1 is human β4GalT1. In some embodiments, the β4GalT1 is at least 85% identical to SEQ ID NO: 8, 37, or 39. In some embodiments, the ST6Gal1 comprises an amino acid sequence that is at least 90% identical to SEQ ID NO:15 or 14.

In some embodiments, the β4GalT1 is bound to the solid support through at least one tag. In some embodiments, the at least one tag is at the N terminus, C terminus, or at both the N terminus and the C terminus. In some embodiments, the at least one tag comprises at least one of a poly(His) tag, chitin binding protein (CBP), maltose binding protein (MBP), glutathione-S-transferase (GST), FLAG-tag, hemagglutinin (HA), Myc, NE-tag, SBP-tag, Strep-tag, calmodulin-tag, Spot-tag, biotin, variants thereof, and combinations thereof. In some embodiments, the at least one tag comprises 3, 4, 5, 6, 7, 8, 9, 10, 11, 12, 13, 14, 15, 16, 17, 18, 19, or 20 histidines (SEQ ID NO: 44).

In some embodiments, the β4GalT1 comprises SEQ ID NO: 8, 37, or 39, or a variant thereof having, 1, 2, 3, 4, 5, 6, 7, 8, 9, or 10 amino acid substitutions, additions, or subtractions. In some embodiments, the ST6Gal1 comprises SEQ ID NO: 15, or a variant thereof having, 1, 2, 3, 4, 5, 6, 7, 8, 9, or 10 amino acid substitutions, additions, or subtractions.

In some embodiments, the β1,4-Galactosyltransferase I (β4GalT1) bound to a solid is separated from the galactosylated IgG antibodies prior to step (b).

In some embodiments, the solid support is a column, array, microarray, or solid phase. In some embodiments, the column, array, microarray, or solid phase comprises a metal (e.g. metal chelate), Nickel (e.g. Ni2+), Cobalt (e.g. Co2+), chitin, maltose, GSH, an antibody or nanobody, a FLAG-binding antibody or nanobody, a HA-binding antibody or nanobody, a Myc-binding antibody or nanobody, an NE-binding antibody or nanobody, streptavidin, biotin, calmodulin, a Spot-binding antibody or nanobody, variants thereof, and combinations thereof.

In some embodiments, the IgG antibodies comprise IgG antibodies isolated from at least 1000 donors. In some embodiments, at least 70% w/w of the IgG antibodies are IgG1 antibodies. In some embodiments, at least 90% of the donor subjects have been exposed to a virus. In some embodiments, about 60%, 65%, 70%, 75%, 80%, or 85% of the branched glycans on the IgG antibodies in the hsIgG preparation have a sialic acid on both the α1,3 branch and the α1,6 branch. In some embodiments, at least 60%, 65%, 70%, 75%, 80%, or 85% of the branched glycans on the Fab domain of the IgG antibodies in the hsIgG preparation have a sialic acid on both the α 1,3 arm and the α 1,6 arm that is connected through a NeuAc-α 2,6-Gal terminal linkage; and at least 60%, 65%, 70%, 75%, 80%, or 85% of the branched glycans on the Fc domain of the IgG antibodies in the hsIgG preparation have a sialic acid on both the α 1,3 arm and the α 1,6 arm that is connected through a NeuAc-α 2,6-Gal terminal linkage.

In hypersialylated IgG at least 70% (e.g., 75%, 80%, 82%, 85%, 87%, 90%, 92%, 94%, 95%, 97%, 98% up to and including 100%) of branched glycans on the Fc region are di-sialylated (i.e., on both the α 1,3 branch and the α 1,6 arm) by way of NeuAc-α 2,6-Gal terminal linkages. In some embodiments, less than 50% (e.g., less than 40%, 30%, 20%, 15%, 10%, 5%, 4%, 3%, 2%, 1%) of branched glycans on the Fc region are mono-sialylated (i.e., sialylated only on the α 1,3 branch or only on the α 1,6 branch) by way of a NeuAc-α 2,6-Gal terminal linkage.

In some embodiments, the polypeptides are derived from plasma, e.g., human plasma. In certain embodiments, the polypeptides are overwhelmingly IgG polypeptides (e.g., IgG1, IgG2, IgG3 or IgG4 or mixtures thereof), although trace amounts of other contain trace amount of other immunoglobulin subclasses can be present.

As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda.

As used herein, the term “constant region” refers to a polypeptide that corresponds to, or is derived from, one or more constant region immunoglobulin domains of an antibody. A constant region can include any or all of the following immunoglobulin domains: a C_{H}1 domain, a hinge region, a C_{H}2 domain, a C_{H}3 domain (derived from an IgA, IgD, IgG, IgE, or IgM), and a C_{H}4 domain (derived from an IgE or IgM).

As used herein, the term “Fc region” refers to a dimer of two “Fc polypeptides,” each “Fc polypeptide” including the constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or the entire flexible hinge N-terminal to these domains. For IgG, “Fc polypeptide” comprises immunoglobulin domains Cgamma2 (Cγ2) and Cgamma3 (Cγ3) and the lower part of the hinge between Cgamma1 (Cγ1) and Cγ2. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, VA). For IgA, Fc polypeptide comprises immunoglobulin domains Calpha2 (Cα2) and Calpha3 (Cα3) and the lower part of the hinge between Calpha1 (Cα1) and Cα2. An Fc region can be synthetic, recombinant, or generated from natural sources such as IVIg.

As used herein, “glycan” is a sugar, which can be monomers or polymers of sugar residues, such as at least three sugars, and can be linear or branched. A “glycan” can include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g., of a polypeptide, glycolipid, proteoglycan, etc.). The term also encompasses free glycans, including glycans that have been cleaved or otherwise released from a glycoconjugate.

As used herein, the term “glycoprotein” refers to a protein that contains a peptide backbone covalently linked to one or more sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. Glycoproteins can contain O-linked sugar moieties and/or N-linked sugar moieties.

As used herein, “IVIg” is a preparation of pooled, polyvalent IgG, including all four IgG subgroups, extracted from plasma of at least 1,000 human donors. IVIg is approved as a plasma protein replacement therapy for immune deficient patients. The level of IVIg Fc glycan sialylation varies among IVIg preparations, but is generally less than 20%. The level of disialylation is generally far lower. As used herein, the term “derived from IVIg” refers to polypeptides which result from manipulation of IVIg. For example, polypeptides purified from IVIg (e.g., enriched for sialylated IgGs or modified IVIg (e.g., IVIg IgGs enzymatically sialylated).

As used herein, an “N-glycosylation site of an Fc polypeptide” refers to an amino acid residue within an Fc polypeptide to which a glycan is N-linked. In some embodiments, an Fc region contains a dimer of Fc polypeptides, and the Fc region comprises two N-glycosylation sites, one on each Fc polypeptide.

As used herein “percent (%) of branched glycans” refers to the number of moles of glycan X relative to total moles of glycans present, wherein X represents the glycan of interest.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents.

“Pharmaceutical preparations” and “pharmaceutical products” can be included in kits containing the preparation or product and instructions for use.

“Pharmaceutical preparations” and “pharmaceutical products” generally refer to compositions in which the final predetermined level of sialylation has been achieved, and which are free of process impurities. To that end, “pharmaceutical preparations” and “pharmaceutical products” are substantially free of ST6Gal sialyltransferase and/or sialic acid donor (e.g., cytidine 5′-monophospho-N-acetyl neuraminic acid) or the byproducts thereof (e.g., cytidine 5′-monophosphate).

“Pharmaceutical preparations” and “pharmaceutical products” are generally substantially free of other components of a cell in which the glycoproteins were produced (e.g., the endoplasmic reticulum or cytoplasmic proteins and RNA), if recombinant.

By “purified” (or “isolated”) refers to a polynucleotide or a polypeptide that is removed or separated from other components present in its natural environment. For example, an isolated polypeptide is one that is separated from other components of a cell in which it was produced (e.g., the endoplasmic reticulum or cytoplasmic proteins and RNA). An isolated polynucleotide is one that is separated from other nuclear components (e.g., histones) and/or from upstream or downstream nucleic acids. An isolated polynucleotide or polypeptide can be at least 60% free, or at least 75% free, or at least 90% free, or at least 95% free from other components present in natural environment of the indicated polynucleotide or polypeptide.

As used herein, the term “sialylated” refers to a glycan having a terminal sialic acid. The term “mono-sialylated” refers to branched glycans having one terminal sialic acid, e.g., on an α1,3 branch or an α1,6 branch. The term “di-sialylated” refers to a branched glycan having a terminal sialic acid on two arms, e.g., both an α1,3 arm and an α1,6 arm.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows a short, branched core oligosaccharide comprising two N-acetylglucosamine and three mannose residues. One of the branches is referred to in the art as the “α 1,3 arm,” and the second branch is referred to as the “α 1,6 arm,”. Squares: N-acetylglucosamine; dark gray circles: mannose; light gray circles: galactose; diamonds: N-acetylneuraminic acid; triangles: fucose.

FIG. 2 shows common Fc glycans present in IVIg. Squares: N-acetylglucosamine; dark gray circles: mannose; light gray circles: galactose; diamonds: N-acetylneuraminic acid; triangles: fucose. FIG. 2 discloses SEQ ID NO: 40.

FIG. 3 shows how immunoglobulins, e.g., IgG antibodies, can be sialylated by carrying out a galactosylation step followed by a sialylation step. Squares: N-acetylglucosamine; dark gray circles: mannose; light gray circles: galactose; diamonds: N-acetylneuraminic acid; triangles: fucose.

FIG. 4 shows a visual representation of SEQ ID NO:38 (amino acids 8-308 of SEQ ID NO: 46) and the corresponding protein structure. The two disulfides are marked in the map, as is the N-glycan. The affinity tag is the His-tag at the C-terminus.

FIG. 5 shows the reaction product of a representative example of the IgG-Fc glycan profile for a reaction starting with IVIg. The left panel is a schematic representation of enzymatic sialylation reaction to transform IgG to hsIgG; the right panel is the IgG Fc glycan profile for the starting IVIg and hsIgG. Bars, from left to right, correspond to IgG1, IgG2/3, and IgG3/4, respectively.

FIG. 6 is bar graph showing relative abundance of the N-glycopeptides following galactosylation. Free/soluble enzyme (1×) is in column 2 in each group, and three different experiments of immobilizing the B4-GalT are in columns 3, 4, and 5 in each group. The starting Immunoglobins are in column 1 in each group.

FIG. 7 shows a schematic of an exemplary hypersialylated IgG preparation. Squares: N-acetylglucosamine; dark gray circles: mannose; light gray circles: galactose; diamonds: N-acetylneuraminic acid; triangles: fucose.

FIG. 8 shows the experimental process for B4-GalT immobilization and analysis.

FIG. 9 shows how enzyme activity was measured.

FIGS. 10A-10D show enzyme immobilization of B4-GalT. FIG. 10A shows attachment of B4-GalT. FIG. 10B shows substrates for immobilization. FIG. 10C shows enzyme activity of free and immobilized enzyme (N=3). FIG. 10D shows enzyme stability at 37° C. over time.

FIGS. 11A-11D show galactosylation of IVIGS using enzyme immobilized B4-GalT. FIG. 11A shows various glycan structures. Squares: N-acetylglucosamine; dark gray circles: mannose; light gray circles: galactose; diamonds: N-acetylneuraminic acid; triangles: fucose. FIG. 11B shows abundant glycan structures typical to IVIG (bars, from left to right: IgG1, IgG2/3). FIG. 11C shows relative abundance of IgG1 glycopeptides after galactosylation with magnetic bead immobilized B4-GalT. 1×=same number of units as free enzyme reaction (bars, from left to right: Free, Mag 1×, Mag 2×). FIG. 11D shows relative abundance of IgG2/G3 N-glycopeptides after galactosylation with magnetic bead immobilized B4-GalT. 1×=same number of units as free enzyme reaction (bars, from left to right: Free, Mag 2×).

FIGS. 12A-12C show B4-GalT immobilized via amine coupling chemistry. FIG. 12A shows attachment of B4-GalT. FIG. 12B shows substrates for immobilization. FIG. 12C shows enzyme activity of free and immobilized enzymes.

FIGS. 13A-13C show B4-GalT immobilization via multi-point epoxy chemistry. FIG. 13A shows attachment of B4-GalT. FIG. 13B shows substrates for immobilization. FIG. 13C shows enzyme activity of free and immobilized enzymes.

### DETAILED DESCRIPTION

Antibodies are glycosylated at conserved positions in the constant regions of their heavy chain and on the Fab domain. For example, human IgG antibodies have a single N-linked glycosylation site at Asn297 of the CH2 domain. Each antibody isotype has a distinct variety of N-linked carbohydrate structures in the constant regions. For human IgG, the core oligosaccharide normally consists of GlcNAc₂Man₃GlcNAc, with differing numbers of outer residues. Variation among individual IgG's can occur via attachment of galactose and/or galactose-sialic acid at one or both terminal GlcNAc or via attachment of a third GlcNAc arm (bisecting GlcNAc).

The present disclosure encompasses, in part, methods for preparing immunoglobulins (e.g., human IgG) having an Fc region having particular levels of branched glycans that are sialylated on both of the arms of the branched glycan (e.g., with a NeuAc-α 2,6-Gal terminal linkage). The levels can be measured on an individual Fc region (e.g., the number of branched glycans that are sialylated on an α1,3 arm, an α1,6 arm, or both, of the branched glycans in the Fc region), or on the overall composition of a preparation of polypeptides (e.g., the number or percentage of branched glycans that are sialylated on an α1,3 arm, an α1,6 arm, or both, of the branched glycans in the Fc region in a preparation of polypeptides).

Naturally derived polypeptides that can be used to prepare hypersialylated IgG include, for example, IgG in human serum (particular human serum pooled from more than 1,000 donors), intravenous immunoglobulin (IVIg) and polypeptides derived from IVIg (e.g., polypeptides purified from IVIg (e.g., enriched for sialylated IgGs) or modified IVIg (e.g., IVIg IgGs enzymatically sialylated).

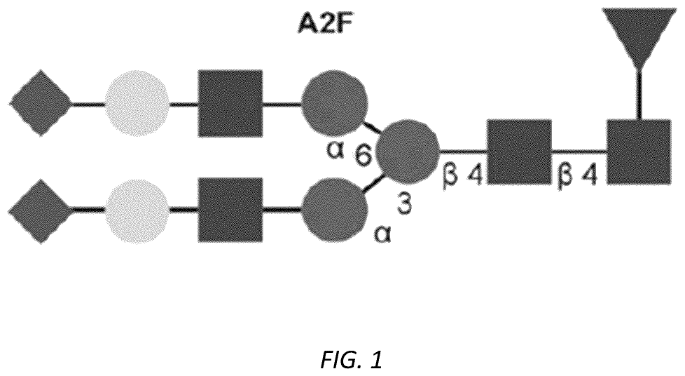

N-linked oligosaccharide chains are added to a protein in the lumen of the endoplasmic reticulum. Specifically, an initial oligosaccharide (typically 14-sugar) is added to the amino group on the side chain of an asparagine residue contained within the target consensus sequence of Asn-X-Ser/Thr, where X may be any amino acid except proline. The structure of this initial oligosaccharide is common to most eukaryotes, and contains three glucose, nine mannose, and two N-acetylglucosamine residues. This initial oligosaccharide chain can be trimmed by specific glycosidase enzymes in the endoplasmic reticulum, resulting in a short, branched core oligosaccharide composed of two N-acetylglucosamine and three mannose residues. One of the branches is referred to in the art as the “α 1,3 arm,” and the second branch is referred to as the “α 1,6 arm,” as shown in FIG. 1.

N-glycans can be subdivided into three distinct groups called “high mannose type,” “hybrid type,” and “complex type,” with a common pentasaccharide core (Man (α 1,6)-(Man(α 1,3))-Man(β 1,4)-GlcpNAc(β 1,4)-GlcpNAc(β 1,N)-Asn) occurring in all three groups.

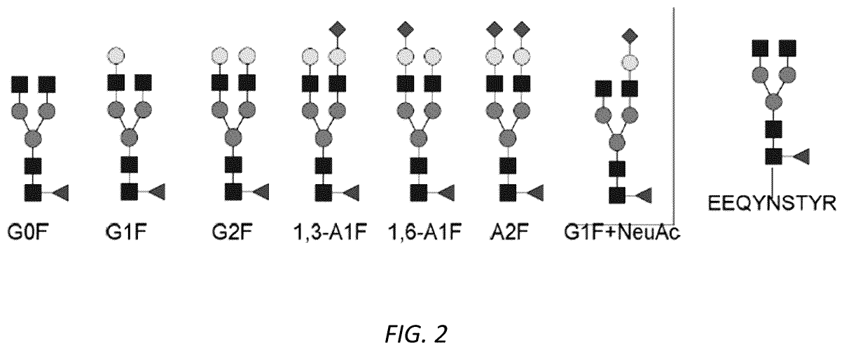

The more common Fc glycans present in IVIg are shown in FIG. 2.

Additionally or alternatively, one or more monosaccharides units of N-acetylglucosamine may be added to the core mannose subunits to form a “complex glycan.” Galactose may be added to the N-acetylglucosamine subunits, and sialic acid subunits may be added to the galactose subunits, resulting in chains that terminate with any of a sialic acid, a galactose or an N-acetylglucosamine residue. Additionally, a fucose residue may be added to an N-acetylglucosamine residue of the core oligosaccharide. Each of these additions is catalyzed by specific glycosyl transferases.

“Hybrid glycans” comprise characteristics of both high-mannose and complex glycans. For example, one branch of a hybrid glycan may comprise primarily or exclusively mannose residues, while another branch may comprise N-acetylglucosamine, sialic acid, galactose, and/or fucose sugars.

Sialic acids are a family of 9-carbon monosaccharides with heterocyclic ring structures. They bear a negative charge via a carboxylic acid group attached to the ring as well as other chemical decorations including N-acetyl and N-glycolyl groups. The two main types of sialyl residues found in polypeptides produced in mammalian expression systems are N-acetyl-neuraminic acid (NeuAc) and N-glycolylneuraminic acid (NeuGc). These usually occur as terminal structures attached to galactose (Gal) residues at the non-reducing termini of both N- and O-linked glycans. The glycosidic linkage configurations for these sialyl groups can be either α 2,3 or α 2,6.

Fc regions are glycosylated at conserved, N-linked glycosylation sites. For example, each heavy chain of an IgG antibody has a single N-linked glycosylation site at Asn297 of the CH2 domain. IgA antibodies have N-linked glycosylation sites within the CH2 and CH3 domains, IgE antibodies have N-linked glycosylation sites within the CH3 domain, and IgM antibodies have N-linked glycosylation sites within the CH1, CH2, CH3, and CH4 domains.

Each antibody isotype has a distinct variety of N-linked carbohydrate structures in the constant regions. For example, IgG has a single N-linked biantennary carbohydrate at Asn297 of the C_{H}2 domain in each Fc polypeptide of the Fc region, which also contains the binding sites for C1q and FcγR. For human IgG, the core oligosaccharide normally consists of GlcNAc2Man3GlcNAc, with differing numbers of outer residues. Variation among individual IgG can occur via attachment of galactose and/or galactose-sialic acid at one or both terminal GlcNAc or via attachment of a third GlcNAc arm (bisecting GlcNAc).

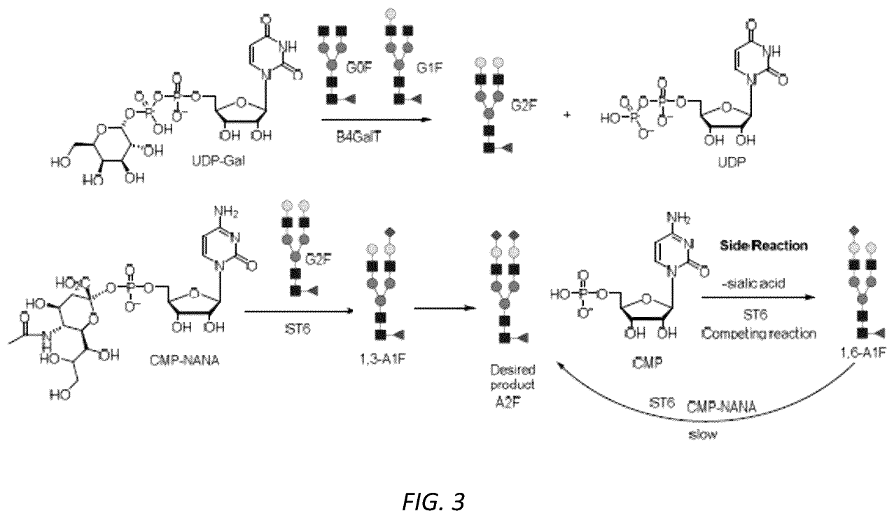

Immunoglobulins, e.g., IgG antibodies, can be sialylated by carrying out a galactosylation step followed by a sialylation step. Beta-1,4-galactosyltransferase 1 (B4GalT) is a Type II Golgi membrane-bound glycoprotein that transfers galactose from uridine 5′-diphosphosegalactose ([[(2R,3S,4R,5R)-5-(2,4-dioxopyrimidin-1-yl)-3,4-dihydroxyoxolan-2-yl]methoxy-hydroxyphosphoryl] [(2R,3R,4S,5R,6R)-3,4,5-trihydroxy-6-(hydroxymethyl)oxan-2-yl] hydrogen phosphate; UDP-Gal) to GlcNAc as a β-1,4 linkage. Alpha-2,6-sialyltransferase 1 (ST6) is a Type II Golgi membrane-bound glycoprotein that transfers sialic acid from cytidine 5′-monophospho-Nacetylneuraminic acid ((2R,4S,5R,6R)-5-acetamido-2-[[(2R,3S,4R,5R)-5-(4-amino-2-oxopyrimidin-1-yl)-3,4-dihydroxyoxolan-2-yl]methoxy-hydroxyphosphoryl]oxy-4-hydroxy-6-(1,2,3-trihydroxypropyl)oxane-2-carboxylic acid; CMP-NANA or CMP-Sialic Acid) to Gal as an α-2,6 linkage. Schematically, the reactions proceed shown in FIG. 3.

Glycans of polypeptides can be evaluated using any methods known in the art. For example, sialylation of glycan compositions (e.g., level of branched glycans that are sialylated on an α1,3 branch and/or an α1,6 branch) can be characterized using methods described in WO2014/179601.

In some embodiments of the hsIgG compositions prepared by the methods described herein, at least 60%, 65%, 70%, 75%, 80%, 85%, or 90% of the branched glycans on the Fc domain have a sialic acid on both the α 1,3 arm and the α 1,6 arm that is connected through a NeuAc-α 2,6-Gal terminal linkage. In addition, in some embodiments, at least 40%, 50%, 60%, 65%, 70%, 75%, 80%, or 85% of the branched glycans on the Fab domain have a sialic acid on both the α 1,3 arm and the α 1,6 arm that is connected through a NeuAc-α 2,6-Gal terminal linkage. Overall, in some embodiments, at least 60%, 65%, 70%, 75%, 80%, 85%, or 90% of the branched glycans have a sialic acid on both the α 1,3 arm and the α 1,6 arm that is connected through a NeuAc-α 2,6-Gal terminal linkage.

### Enzymes

Beta-1,4-galactosyltransferase (B4GalT), e.g., human B4GalT, e.g., human B4Galt1, as well as orthologs, mutants, and variants thereof, including enzymatically active portions of beta-1,4-galactosyltransferase (B4GalT), e.g., human B4GalT, e.g., human B4Galt1, as well as orthologs, mutants, and variants thereof, along with fusion proteins and polypeptides comprising the same are suitable for use in the methods described herein. B4Galt1 is one of seven beta-1,4-galactosyltransferase (beta4GalT) genes that each encode type II membrane-bound glycoproteins that appear to have exclusive specificity for the donor substrate UDP-galactose; all transfer galactose in a beta1,4 linkage to similar acceptor sugars: GlcNAc, Glc, and Xyl. B4Galt1 adds galactose to N-acetylglucosamine residues that are either monosaccharides or the nonreducing ends of glycoprotein carbohydrate chains. B4GalT1 is also called GGTB2. Four alternative transcripts encoding four isoforms of B4GALT1 (NCBI Gene ID 2683) are described in Table 1.

TABLE 1
Human B4GALT1 isoforms

| | | | SEQ | Length | |
|---|---|---|---|---|---|
| Transcript | Length (nt) | Protein | ID NO: | (aa) | Isoform |
| NM_001497.4 | 4176 | NP_001488.2 | 1 | 398 | 1 |
| NM_001378495.1 | 3999 | NP_001365424.1 | 2 | 385 | 2 |
| NM_001378496.1 | 4053 | NP_001365425.1 | 3 | 357 | 3 |
| NM_001378497.1 | 1520 | NP_001365426.1 | 4 | 225 | 4 |

>NP_001488.2 B4GALT1 [organism = Homo sapiens]
[GeneID = 2683] [isoform = 1]
(SEQ ID NO: 1)
MRLREPLLSGSAAMPGASLQRACRLLVAVCALHLGVTLVYYLAGRDLS
RLPQLVGVSTPLQGGSNSAAAIGQSSGELRTGGARPPPPLGASSQPRP
GGDSSPVVDSGPGPASNLTSVPVPHTTALSLPACPEESPLLVGPMLIE
FNMPVDLELVAKQNPNVKMGGRYAPRDCVSPHKVAIIIPFRNRQEHLK
YWLYYLHPVLQRQQLDYGIYVINQAGDTIFNRAKLLNVGFQEALKDYD
YTCFVFSDVDLIPMNDHNAYRCFSQPRHISVAMDKFGFSLPYVQYFGG
VSALSKQQFLTINGFPNNYWGWGGEDDDIFNRLVFRGMSISRPNAVVG
RCRMIRHSRDKKNEPNPQRFDRIAHTKETMLSDGLNSLTYQVLDVQRY
PLYTQITVDIGTPS
>NP_001365424.1 B4GALT1 [organism = Homo sapiens]
[GeneID = 2683] [isoform = 2]
(SEQ ID NO: 2)
MPGASLQRACRLLVAVCALHLGVTLVYYLAGRDLSRLPQLVGVSTPLQ
GGSNSAAAIGQSSGELRTGGARPPPPLGASSQPRPGGDSSPVVDSGPG
PASNLTSVPVPHTTALSLPACPEESPLLVGPMLIEFNMPVDLELVAKQ
NPNVKMGGRYAPRDCVSPHKVAIIIPFRNRQEHLKYWLYYLHPVLQRQ
QLDYGIYVINQAGDTIFNRAKLLNVGFQEALKDYDYTCFVFSDVDLIP
MNDHNAYRCFSQPRHISVAMDKFGFSLPYVQYFGGVSALSKQQFLTIN
GFPNNYWGWGGEDDDIFNRLVFRGMSISRPNAVVGRCRMIRHSRDKKN
EPNPQRFDRIAHTKETMLSDGLNSLTYQVLDVQRYPLYTQITVDIGTP
S
>NP_001365425.1 B4GALT1 [organism = Homo sapiens]
[GeneID = 2683] [isoform = 3]
(SEQ ID NO: 3)
MRLREPLLSGSAAMPGASLQRACRLLVAVCALHLGVTLVYYLAGRDLS
RLPQLVGVSTPLQGGSNSAAAIGQSSGELRTGGARPPPPLGASSQPRP
GGDSSPVVDSGPGPASNLTSVPVPHTTALSLPACPEESPLLVGPMLIE
FNMPVDLELVAKQNPNVKMGGRYAPRDCVSPHKVAIIIPFRNRQEHLK
YWLYYLHPVLQRQQLDYGIYVINQAGDTIFNRAKLLNVGFQEALKDYD
YTCFVFSDVDLIPMNDHNAYRCFSQPRHISVAMDKFGFRLVFRGMSIS
RPNAVVGRCRMIRHSRDKKNEPNPQRFDRIAHTKETMLSDGLNSLTYQ
VLDVQRYPLYTQITVDIGTPS
>NP_001365426.1 B4GALT1 [organism = Homo sapiens]
[GeneID = 2683] [isoform = 4]
(SEQ ID NO: 4)
MRLREPLLSGSAAMPGASLQRACRLLVAVCALHLGVTLVYYLAGRDLS
RLPQLVGVSTPLQGGSNSAAAIGQSSGELRTGGARPPPPLGASSQPRP
GGDSSPVVDSGPGPASNLTSVPVPHTTALSLPACPEESPLLVGPMLIE
FNMPVDLELVAKQNPNVKMGGRYAPRDCVSPHKVAIIIPFRNRQEHLK
YWLYYLHPVLQRQQLDYGIYVINQYEKIRRLLW

TABLE 2
Topology of B4GALT1 isoform 1 (SEQ ID NO: 1)

| Feature | AAs | Description | Length | Sequence | SEQ ID NO: |
|---|---|---|---|---|---|
| Topological | 1-24 | Cytoplasmic | 9 | MRLREPLLSGSAAMPGASLQR | 5 |
| domain | | | | ACR | |
| Transmembrane | 25-44 | Helical; | 17 | LLVAVCALHLGVTLVYYLAG | 6 |
| | | Signal- | | | |
| | | anchor for | | | |
| | | type II | | | |
| | | membrane | | | |
| | | protein | | | |
| Topological | 45-398 | Lumenal | 380 | RDLSRLPQLVGVSTPLQGGSN | 7 |
| domain | | | | SAAAIGQSSGELRTGGARPPP | |
| | | | | PLGASSQPRPGGDSSPVVDSG | |
| | | | | PGPASNLTSVPVPHTTALSLP | |
| | | | | ACPEESPLLVGPMLIEFNMPV | |
| | | | | DLELVAKQNPNVKMGGRYAPR | |
| | | | | DCVSPHKVAIIIPFRNRQEHL | |
| | | | | KYWLYYLHPVLQRQQLDYGIY | |
| | | | | VINQAGDTIFNRAKLLNVGFQ | |
| | | | | EALKDYDYTCFVFSDVDLIPM | |
| | | | | NDHNAYRCFSQPRHISVAMDK | |
| | | | | FGFSLPYVQYFGGVSALSKQQ | |
| | | | | FLTINGFPNNYWGWGGEDDDI | |
| | | | | FNRLVFRGMSISRPNAVVGRC | |
| | | | | RMIRHSRDKKNEPNPQRFDRI | |
| | | | | AHTKETMLSDGLNSLTYQVLD | |
| | | | | VQRYPLYTQITVDIGTPS | |

TABLE 3
Binding sites of B4GALT1 isoform 1 (SEQ ID NO:1)

| Position(s) | Description | Reference(s) |
|---|---|---|
| 250 | Metal binding; | |
| | Manganese | |
| 310 | Binding site; | “Structural snapshots of beta-1,4- |
| | UDP-alpha-D- | galactosyltransferase-I along the kinetic pathway.” |
| | galactose | Ramakrishnan B., Ramasamy V., Qasba P. K. |
| | | J. Mol. Biol. 357:1619-1633(2006) |
| 343 | Metal binding; | |
| | Manganese; via | |
| | tele nitrogen | |
| 355 | Binding site; N- | “Oligosaccharide preferences of beta1,4- |
| | acetyl-D- | galactosyltransferase-I: crystal structures of |
| | glucosamine | Met340His mutant of human beta1,4- |
| | | galactosyltransferase-I with a pentasaccharide and |
| | | trisaccharides of the N-glycan moiety.” |
| | | Ramasamy V., Ramakrishnan B., Boeggeman E., |
| | | Ratner D. M., Seeberger P. H., Qasba P. K. |
| | | J. Mol. Biol. 353:53-67(2005) |
| | | “Deoxygenated disaccharide analogs as specific |
| | | inhibitors of beta1-4-galactosyltransferase 1 and |
| | | selectin-mediated tumor metastasis.” |
| | | Brown J. R., Yang F., Sinha A., Ramakrishnan B., Tor |
| | | Y., Qasba P. K., Esko J. D. |
| | | J. Biol. Chem. 284:4952-4959(2009) |

TABLE 4
Post Translational Amino Acid Modifications of B4GALT1 isoform 1 (SEQ
ID NO: 1)

| Feature key | Position(s) | Description | Reference(s) |
|---|---|---|---|
| Glycosylation | 113 | N-linked | |
| | | (GlcNAc . . .) | |
| | | asparagine | |
| Disulfide | 130 ↔ 172 | | “Oligosaccharide preferences of beta1,4- |
| bond | | | galactosyltransferase-I: crystal structures of |
| | | | Met340His mutant of human beta1,4- |
| Disulfide | 243 ↔ 262 | | galactosyltransferase-I with a |
| bond | | | pentasaccharide and trisaccharides of the N- |
| | | | glycan moiety.” |
| | | | Ramasamy V., Ramakrishnan B., |
| | | | Boeggeman E., Ratner D. M., Seeberger |
| | | | P.H., Qasba P. K. |
| | | | J. Mol. Biol. 353:53-67(2005) |
| | | | “Structural snapshots of beta-1,4- |
| | | | galactosyltransferase-I along the kinetic |
| | | | pathway.” |
| | | | Ramakrishnan B., Ramasamy V., Qasba |
| | | | P. K. |
| | | | J. Mol. Biol. 357:1619-1633(2006) |

The soluble form of B4GalT1 derives from the membrane form by proteolytic processing. The cleavage site is at positions 77-78 of B4GALT1 isoform 1 (SEQ ID NO:1).

In some embodiments, one or more of the amino acids of the B4GalT1 corresponding to amino acids 113, 130, 172, 243, 250, 262, 310, 343, or 355 of B4GALT1 isoform 1 (SEQ ID NO:1) is conserved as compared to (SEQ ID NO:1).

In some embodiments, the enzyme is an enzymatically active portion of, e.g., B4GalT1. In some embodiments, the enzyme is an enzymatically active portion of B4GALT1 isoform 1 (SEQ ID NO:1), or an ortholog, mutant, or variant of SEQ ID NO:1. In some embodiments, the enzyme is an enzymatically active portion of B4GALT1 isoform 2 (SEQ ID NO:2), or an ortholog, mutant, or variant of SEQ ID NO:2. In some embodiments, the enzyme is an enzymatically active portion of B4GALT1 isoform 3 (SEQ ID NO:3), or an ortholog, mutant, or variant of SEQ ID NO:3. In some embodiments, the enzyme is an enzymatically active portion of B4GALT1 isoform 4 (SEQ ID NO:4), or an ortholog, mutant, or variant of SEQ ID NO:4.

In some embodiments, the enzymatically active portion of B4GalT1 does not comprise a cytoplasmic domain, e.g., SEQ ID NO:5. In some embodiments, the enzymatically active portion of B4GalT1 does not comprise a transmembrane domain, e.g., SEQ ID NO:6. In some embodiments, the enzymatically active portion of B4GalT1 does not comprise a cytoplasmic domain, e.g., SEQ ID NO:5 or a transmembrane domain, e.g., SEQ ID NO:6.

In some embodiments, the enzymatically active portion of B4GalT1 comprises all or a portion of a luminal domain, e.g., SEQ ID NO:7, or an ortholog, mutants, or variants thereof.

In some embodiments, the enzymatically active portion of B4GalT1 comprises amino acids 109-398 of SEQ ID NO:1, or an ortholog, mutants, or variants thereof. In some embodiments, the enzymatically active portion of B4GalT1 consists of SEQ ID NO:1, or an ortholog, mutant, or variant of SEQ ID NO:1.

A suitable functional portion of an B4GalT1 can comprise or consist of an amino acid sequence that is at least 80% (85%, 90%, 95%, 98% or 100%) identical to SEQ ID NO:8.

SEQ ID NO: 8
GPASNLTSVPVPHTTALSLPACPEESPLLVGPMLIEFNMPVDLELVAKQ
NPNVKMGGRYAPRDCVSPHKVAIIIPFRNRQEHLKYWLYYLHPVLQRQQ
LDYGIYVINQAGDTIFNRAKLLNVGFQEALKDYDYTCFVFSDVDLIPMN
DHNAYRCFSQPRHISVAMDKFGFSLPYVQYFGGVSALSKQQFLTINGFP
NNYWGWGGEDDDIFNRLVFRGMSISRPNAVVGRCRMIRHSRDKKNEPNP
QRFDRIAHTKETMLSDGLNSLTYQVLDVQRYPLYTQITVDIGTPS

ST6Gal1, e.g., human ST6Gal1, as well as orthologs, mutants, and variants thereof, including enzymatically active portions of ST6Gal1, e.g., human ST6Gal1, as well as orthologs, mutants, and variants thereof, along with fusion proteins and polypeptides comprising the same, are suitable for use in the methods described herein. ST6GAL1, β-galactoside α-2,6-sialyltransferase 1, transfers sialic acid from CMP-sialic acid to the Galβ1→4GlcNAc structure on glycoproteins, such as asialofetuin and asialo-a1-acid glycoprotein. ST6Gal1 is also called as ST6N or SIAT1. Four alternative transcripts encoding two isoforms of ST6GAL1 (NCBI Gene ID 6480) are described in Table 1.

TABLE 1
Human ST6GAL1 isoforms

| | | | SEQ | | |
|---|---|---|---|---|---|
| | Length | | ID | Length | |
| Transcript | (nt) | Protein | NO: | (aa) | Isoform |
| NM_173216.2 | 4604 | NP_775323.1 | 9 | 406 | a |
| NM_173217.2 | 3947 | NP_775324.1 | 10 | 175 | b |
| NM_003032.3 | 4303 | NP_003023.1 | 9 | 406 | a |
| NM_001353916.2 | 4177 | NP_001340845.1 | 9 | 406 | a |

>NP_001340845.1 (NP_003023.1, NP_775323.1) ST6GAL1
[organism = Homo sapiens] [GeneID = 6480]
[isoform = a]
(SEQ ID NO : 9)
MIHTNLKKKFSCCVLVFLLFAVICVWKEKKKGSYYDSFKLQTKEFQVLKS
LGKLAMGSDSQSVSSSSTQDPHRGRQTLGSLRGLAKAKPEASFQVWNKDS
SSKNLIPRLQKIWKNYLSMNKYKVSYKGPGPGIKFSAEALRCHLRDHVNV
SMVEVTDFPFNTSEWEGYLPKESIRTKAGPWGRCAVVSSAGSLKSSQLGR
EIDDHDAVLRFNGAPTANFQQDVGTKTTIRLMNSQLVTTEKRFLKDSLYN
EGILIVWDPSVYHSDIPKWYQNPDYNFFNNYKTYRKLHPNQPFYILKPQM
PWELWDILQEISPEEIQPNPPSSGMLGIIIMMTLCDQVDIYEFLPSKRKT
DVCYYYQKFFDSACTMGAYHPLLYEKNLVKHLNQGTDEDIYLLGKATLPG
FRTIHC
>NP_775324.1 ST6GAL1 [organism = Homo sapiens]
[GeneID = 6480] [isoform = b]
(SEQ ID NO: 10)
MNSQLVTTEKRFLKDSLYNEGILIVWDPSVYHSDIPKWYQNPDYNFFNNY
KTYRKLHPNQPFYILKPQMPWELWDILQEISPEEIQPNPPSSGMLGIIIM
MTLCDQVDIYEFLPSKRKTDVCYYYQKFFDSACTMGAYHPLLYEKNLVKH
LNQGTDEDIYLLGKATLPGFRTIHC

TABLE 2
Topology of ST6Gal1 isoform a (SEQ ID NO: 28)

| Feature | AAs | Description | Length | Sequence | SEQ ID NO: |
|---|---|---|---|---|---|
| Topological | 1-9 | Cytoplasmic | 9 | MIHTNLKKK | 11 |
| domain | | | | | |
| Transmembrane | 10-26 | Helical; | 17 | FSCCVLVFLLFAVICVW | 12 |
| | | Signal- | | | |
| | | anchor for | | | |
| | | type II | | | |
| | | membrane | | | |
| | | protein | | | |
| Topological | 27-406 | Lumenal | 380 | KEKKKGSYYDSFKLQTKEFQVLKS | 13 |
| domain | | | | LGKLAMGSDSQSVSSSSTQDPHRG | |
| | | | | RQTLGSLRGLAKAKPEASFQVWNK | |
| | | | | DSSSKNLIPRLQKIWKNYLSMNKY | |
| | | | | KVSYKGPGPGIKFSAEALRCHLRD | |
| | | | | HVNVSMVEVTDFPFNTSEWEGYLP | |
| | | | | KESIRTKAGPWGRCAVVSSAGSLK | |
| | | | | SSQLGREIDDHDAVLRFNGAPTAN | |
| | | | | FQQDVGTKTTIRLMNSQLVTTEKR | |
| | | | | FLKDSLYNEGILIVWDPSVYHSDI | |
| | | | | PKWYQNPDYNFFNNYKTYRKLHPN | |
| | | | | QPFYILKPQMPWELWDILQEISPE | |
| | | | | EIQPNPPSSGMLGIIIMMTLCDQV | |
| | | | | DIYEFLPSKRKTDVCYYYQKFFDS | |
| | | | | ACTMGAYHPLLYEKNLVKHLNQGT | |
| | | | | DEDIYLLGKATLPGFRTIHC | |

TABLE 3
Binding sites of ST6Gal1 isoform a (SEQ ID NO: 28)

| Position(s) | Description | Reference(s) |
|---|---|---|
| 189 | Substrate; via | “The structure of human alpha-2,6-sialyltransferase |
| | amide nitrogen | reveals the binding mode of complex glycans.” |
| 212 | Substrate | Kuhn B., Benz J., Greif M., Engel A. M., Sobek H., |
| 233 | Substrate | Rudolph M.G. Acta Crystallogr. D 69:1826- |
| 353 | Substrate; via | 1838(2013) |
| | carbonyl oxygen | |
| 354 | Substrate | |
| 365 | Substrate | |
| 369 | Substrate | |
| 370 | Substrate | “The structure of human alpha-2,6-sialyltransferase |
| | | reveals the binding mode of complex glycans.” |
| 376 | Substrate | Kuhn B., Benz J., Greif M., Engel A. M., Sobek H., |
| | | Rudolph M. G. Acta Crystallogr. D 69:1826- |
| | | 1838(2013) |

TABLE 4
Post Translational Amino Acid Modifications of ST6Gal1 isoform a (SEQ
ID NO: 28)

| Feature key | Position(s) | Description | Reference(s) |
|---|---|---|---|
| Disulfide | 142 ↔ 406 | | “The structure of human alpha-2,6- |
| bond | | | sialyltransferase reveals the binding |
| | | | mode of complex glycans.” |
| | | | Kuhn B., Benz J., Greif M., Engel |
| | | | A.M., Sobek H., Rudolph M. G. |
| | | | Acta Crystallogr. D 69:1826- |
| | | | 1838(2013) |
| Glycosylation | 149 | N-linked | “Glycoproteomics analysis of human |
| | | (GlcNAc . . .) | liver tissue by combination of multiple |
| | | asparagine | enzyme digestion and hydrazide |
| | | | chemistry.” |
| | | | Chen R., Jiang X., Sun D., Han G., |
| | | | Wang F., Ye M., Wang L., Zou H. |
| | | | J. Proteome Res. 8:651-661(2009); and |
| | | | “The structure of human alpha-2,6- |
| | | | sialyltransferase reveals the binding |
| | | | mode of complex glycans.” |
| | | | Kuhn B., Benz J., Greif M., Engel |
| | | | A. M., Sobek H., Rudolph M. G. |
| | | | Acta Crystallogr. D 69:1826- |
| | | | 1838(2013) |
| Glycosylation | 161 | N-linked | “Glycoproteomics analysis of human |
| | | (GlcNAc . . .) | liver tissue by combination of multiple |
| | | asparagine | enzyme digestion and hydrazide |
| | | | chemistry.” |
| | | | Chen R., Jiang X., Sun D., Han G., |
| | | | Wang F., Ye M., Wang L., Zou H. |
| | | | J. Proteome Res. |
| | | | 8:651-661(2009) |
| Disulfide | 184 ↔ 335 | | “The structure of human alpha-2,6- |
| bond | | | sialyltransferase reveals the binding |
| | | | mode of complex glycans.” |
| | | | Kuhn B., Benz J., Greif M., Engel |
| | | | A. M., Sobek H., Rudolph M. G. |
| | | | Acta Crystallogr. D 69:1826- |
| | | | 1838(2013) |
| Disulfide | 353 ↔ 364 | | “The structure of human alpha-2,6- |
| bond | | | sialyltransferase reveals the binding |
| | | | mode of complex glycans.” |
| | | | Kuhn B., Benz J., Greif M., Engel |
| | | | A. M., Sobek H., Rudolph M. G. |
| | | | Acta Crystallogr. D 69:1826- |
| | | | 1838(2013) |
| Modified | 369 | Phosphotyrosine | “Quantitative phosphoproteomic |
| residue | | | analysis of T cell receptor signaling |
| | | | reveals system-wide modulation of |
| | | | protein-protein interactions.” |
| | | | Mayya V., Lundgren D. H., Hwang S.-I., |
| | | | Rezaul K., Wu L., Eng J. K., Rodionov |
| | | | V., Han D. K. |
| | | | Sci. Signal. 2:RA46-RA46(2009) |

The soluble form of ST6Gal1 derives from the membrane form by proteolytic processing.

In some embodiments, one or more of the amino acids of the ST6Gal1 corresponding to amino acids 142, 149, 161, 184, 189, 212, 233, 335, 353, 354, 364, 365, 369, 370, 376, or 406 of ST6Gal1 isoform a (SEQ ID NO:9) is conserved as compared to SEQ ID NO:9.

Also provided herein is an enzymatically active portion of, e.g., ST6Gal1. In some embodiments, the enzyme is an enzymatically active portion of STG6Gal1 isoform a (SEQ ID NO:9), or an ortholog, mutant, or variant of SEQ ID NO:9. In some embodiments, the enzyme is an enzymatically active portion of STG6Gal1 isoform b (SEQ ID NO:10), or an ortholog, mutant, or variant of SEQ ID NO:10.

In some embodiments, the enzymatically active portion of ST6Gal1 does not comprise a cytoplasmic domain, e.g., SEQ ID NO:11. In some embodiments, the enzymatically active portion of ST6Gal1 does not comprise a transmembrane domain, e.g., SEQ ID NO:12. In some embodiments, the enzymatically active portion of ST6Gal1 does not comprise a cytoplasmic domain, e.g., SEQ ID NO:11 or a transmembrane domain, e.g., SEQ ID NO:12.

In some embodiments, the enzymatically active portion of ST6Gal1 comprises all or a portion of a luminal domain, e.g., SEQ ID NO:13, or an ortholog, mutants, or variants thereof.

In some embodiments, the enzymatically active portion of ST6Gal1 comprises amino acids 87-406 of SEQ ID NO:9 (SEQ ID NO:14), or an ortholog, mutants, or variants thereof. In some embodiments, the enzymatically active portion of ST6Gal1 consists of SEQ ID NO:4, or an ortholog, mutant, or variant of SEQ ID NO:4.

A suitable functional portion of an ST6Gal1 can comprise or consist of an amino acid sequence that is at least 80% (85%, 90%, 95%, 98% or 100%) identical to SEQ ID NO:14.

SEQ ID NO: 14
AKPEASFQVWNKDSSSKNLIPRLQKIWKNYLSMNKYKVSYKGPGPGIKF
SAEALRCHLRDHVNVSMVEVTDFPFNTSEWEGYLPKESIRTKAGPWGRC
AVVSSAGSLKSSQLGREIDDHDAVLRFNGAPTANFQQDVGTKTTIRLMN
SQLVTTEKRFLKDSLYNEGILIVWDPSVYHSDIPKWYQNPDYNFFNNYK
TYRKLHPNQPFYILKPQMPWELWDILQEISPEEIQPNPPSSGMLGIIIM
MTLCDQVDIYEFLPSKRKTDVCYYYQKFFDSACTMGAYHPLLYEKNLVK
HLNQGTDEDIYLLGKATLPGFRTIHC

In some embodiments, the ST6Gal1 comprises or consists of SEQ ID NO:14, the portion of SEQ ID NO:14 from amino acid 4 to 320, or the portion of SEQ ID NO:14 from amino acid 5 to 320.

Also suitable for use in the methods described herein is an amino acid sequence that is at least 80% (85%, 90%, 95%, 98% or 100%) identical to SEQ ID NO:15.

SEQ ID NO: 15
gssplldmlehhhhhhhhmAKPEASFQVWNKDSSSKNLIPRLQKIWKNY
LSMNKYKVSYKGPGPGIKFSAEALRCHLRDHVNVSMVEVTDFPFNTSEW
EGYLPKESIRTKAGPWGRCAVVSSAGSLKSSQLGREIDDHDAVLRFNGA
PTANFQQDVGTKTTIRLMNSQLVTTEKRFLKDSLYNEGILIVWDPSVYH
SDIPKWYQNPDYNFFNNYKTYRKLHPNQPFYILKPQMPWELWDILQEIS
PEEIQPNPPSSGMLGIIIMMTLCDQVDIYEFLPSKRKTDVCYYYQKFFD
SACTMGAYHPLLYEKNLVKHLNQGTDEDIYLLGKATLPGFRTIHC

### Variants

In some embodiments, the enzyme(s) described herein are at least 80%, e.g., at least 85%, 90%, 95%, 98%, or 100% identical to the amino acid sequence of an exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains desired activity of the parent, e.g., β-galactoside α-2,6-sialyltransferase activity or β-1,4-galactosyltransferase activity.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

### B4GalT Immobilization

In some embodiments, the protein(s) comprising enzyme(s) or portions thereof as described herein are immobilized on a surface, e.g., a solid support.

Methods for protein immobilization, including both covalent and non-covalent approaches, are known and described in the art.

Covalent approaches, such as enzymatic approaches (e.g., sortase A), enzyme self-labeling (e.g., SNAP-tag, HaloTag, and CLIP-tag) chemical approaches (e.g., oxime ligation, Cu(I)-catalyzed axide-alkyne cycoloaddition (CuAAC) reaction, strain-promoted azide-alkyne cycloaddition (SPAAC) reaction, strain-promoted alkyne-nitrone cycloaddition (SPANC) reaction, and inverse electron-demand Diels-Alder reaction (IEDDA) reaction). See, e.g., Meldal and Schoffelen, “Recent Advances in Covalent, Site-Specific Protein Immobilization,” F1000Research 216, 5(F1000 Faculty Rev):2303.

In some embodiments, the protein is immobilized via a non-covalent approach (affinity-mediated mobilization) such as the use of protein A or G for binding of antibodies, peptide tags such as polyhistidine, protein tags such as maltose-binding protein and glutathione-S-transferase, DNA-directed immobilization, or the biotin-streptavidin interaction pair. See e.g., Steen et al., “Protein engineering for directed immobilization,” Bioconjug Chem. 2013; 24(11):1761-77; Liu et al., “Oriented immobilization of proteins on solid supports for use in biosensors and biochips: a review,” Microchim Acta 2016; 183:1-19; Sapsford et al., “Functionalizing nanoparticles with biological molecules: developing chemistries that facilitate nanotechnology,” Chem Rev. 2013; 113(3):1904-2074; Benes̆ová et al., “Affinity Interactions as a Tool for Protein Immobilization,” In: Magdeldin S, editor. Affinity Chromatography: InTech. 2012; 29-46; Trilling et al., “Antibody orientation on biosensor surfaces: a minireview,” Analyst 2013; 138(6):1619-27; and Meyer et al., “Advances in DNA-directed immobilization,” Curr Opin Chem Biol. 2014; 18:8-15.

### Polypeptides

Thus, also provided herein are polypeptides comprising: i) a B4GalT enzyme (e.g., a Beta-1,4-galactosyltransferase (B4GalT), e.g., human B4GalT, e.g., human B4Galt1, or an ortholog, mutants, or variants of Beta-1,4-galactosyltransferase (B4GalT), e.g., human B4GalT, e.g., human B4Galt1, including enzymatically active portions of beta-1,4-galactosyltransferase (B4GalT), e.g., human B4GalT, e.g., human B4Galt1, as well as orthologs, mutants, and variants of an enzymatically active portions of beta-1,4-galactosyltransferase (B4GalT), e.g., human B4GalT, e.g., human B4Galt1); and ii) at least one affinity tag.

In some embodiments, the at least one tag is at the N terminus, C terminus, or at both the N terminus and the C terminus.

In some embodiments, the affinity tag is selected from the group consisting of polyhistidine, chitin binding protein (CBP) (e.g., KRRWKKNFIAVSAANRFKKISSSGAL, SEQ ID NO:16), glutathione S-transferase (GST) (e.g., MSPILGYWKIKGLVQPTRLLLEYLEEKYEEHLYERDEGDKWRNKKFELGLEF PNLPYYIDGDVKLTQSMAIIRYIADKHNMLGGCPKERAEISMLEGAVLDIRYG VSRIAYSKDFETLKVDFLSKLPEMLKMFEDRLCHKTYLNGDHVTHPDFMLYD ALDVVLYMDPMCLDAFPKLVCFKKRIEAIPQIDKYLKSSKYIAWPLQGWQAT FGGGDHPPKSD, SEQ ID NO:17), maltose-binding protein (MBP) (e.g., MGSSHHHHHHSSGLVPRGSHMGSMKIEEGKLVIWINGDKGYNGLAEVGKKF EKDTGIKVTVEHPDKLEEKFPQVAATGDGPDIIFWAHDRFGGYAQSGLLAEIT PDKAFQDKLYPFTWDAVRYNGKLIAYPIAVEALSLIYNKDLLPNPPKTWEEIP ALDKELKAKGKSALMFNLQEPYFTWPLIAADGGYAFKYENGKYDIKDVGVD NAGAKAGLTFLVDLIKNKHMNADTDYSIAEAAFNKGETAMTINGPWAWSNI DTSKVNYGVTVLPTFKGQPSKPFVGVLSAGINAASPNKELAKEFLENYLLTDE GLEAVNKDKPLGAVALKSYEEELAKDPRIAATMENAQKGEIMPNIPQMSAF WYAVRTAVINAASGRQTVDEALKDAQTNSSSLGIEGR, SEQ ID NO:18), hemagglutinin (HA) (e.g., YPYDVPDYA, SEQ ID NO:19), Myc (e.g., EQKLISEEDL, SEQ ID NO:20), streptavidin-binding peptide (SBP) (e.g., MDEKTTGWRGGHVVEGLAGELEQLRARLEHHPQGQREP, SEQ ID NO:21), calmodulin-tag (e.g., MADQLTEEQIAEFKEAFSLFDKDGDGTITTKELGTVMRSLGQNPTEAELQDMI NEVDADGNGTIDFPEFLTMMARKMKDTDSEEEIREAFRVFDKDGNGYISAAE LRHVMTNLGEKLTDEEVDEMIREADIDGDGQVNYEEFVQMMTAKGSMGWD LTVKMLAGNEFQVSLSSSMSVSELKAQITQKIGVHAFQQRLAVHPSGVALQD RVPLASQGLGPGSTVLLVVDKCDEPLNILVRNNKGRSSTYEVRLTQTVAHLK QQVSGLEGVQDDLFWLTFEGKPLEDQLPLGEYGLKPLSTVFMNLRLRGG, SEQ ID NO:22), Spot-tag (e.g., PDRVRAVSHWSS, SEQ ID NO:23), a streptavidin tag (e.g., Strep-Tag®, e.g., Trp-Ser-His-Pro-Gln-Phe-Glu-Lys (SEQ ID NO:24)), FLAG-tag (e.g., DYKDDDDK (SEQ ID NO:25) biotin, as well as variants thereof and combinations of all of the foregoing.

In some embodiments, the affinity tag is a polyhistidine tag. In some embodiments, the polyhistidine tag is selected from the group consisting of HHHH (SEQ ID NO:26), HHHHH (SEQ ID NO:27), HHHHHH, (SEQ ID NO:28), HHHHHHH (SEQ ID NO:29), HHHHHHHH (SEQ ID NO:30), HHHHHHHHH (SEQ ID NO:31), and HHHHHHHHHH (SEQ ID NO:32). In some embodiments, the at least one tag comprises 3, 4, 5, 6, 7, 8, 9, 10, 11, 12, 13, 14, 15, 16, 17, 18, 19, or 20 histidines (SEQ ID NO: 44).

In some embodiments, the affinity tag is situated towards the N-terminal side of the enzyme. In some embodiments, the affinity tag is situated towards the C-terminal side of the enzyme.

Additional tags are known in the art and can be used for the purpose of immobilizing the β4GalT1 to a solid support (e.g. resin, column, array, etc.). In some embodiments, these additional tags may be paired with known binding agents attached to the solid support such that the tagged β4GalT1 binds to the solid support.

In some embodiments, the polypeptide further comprises a cleavage sequence or spacer sequence between the enzyme and the affinity tag (e.g., situated towards the C-terminal side of the enzyme and towards the N-terminal side of the affinity tag). In some embodiments, the spacer sequence is 1, 2, 3, 4, 5, 6, 7, 8, 9, 10, 11, 12, 13, 14, 15, 16, 17, 18, 19, or 20 amino acids long. In some embodiments, the spacer sequence is at least 3, 4, 5, 6, 7, 8, 9, 10, 11, 12, 13, 14, 15, 16, 17, 18, 19, 20, 21, 22, 23, 24, 25, 26, 27, 28, 29 or 30 amino acids long. In some embodiments, the cleavage or spacer sequence is at least 3 amino acids long.

In some embodiments, the spacer sequence comprises or consists of PRD (SEQ ID NO:33). In some embodiments spacer sequence comprises PGG (SEQ ID NO:34).

A suitable B4GalT with a C-terminal spacer sequence that is suitable for use in the methods described herein, therefore, can comprise an amino acid sequence that is at least 80% (85%, 90%, 95%, 98% or 100%) identical to SEQ ID NO:35 or SEQ ID NO:36

SEQ ID NO: 35
GPASNLTSVPVPHTTALSLPACPEESPLLVGPMLIEFNMPVDLELVAKQ
NPNVKMGGRYAPRDCVSPHKVAIIIPFRNRQEHLKYWLYYLHPVLQRQQ
LDYGIYVINQAGDTIFNRAKLLNVGFQEALKDYDYTCFVFSDVDLIPMN
DHNAYRCFSQPRHISVAMDKFGFSLPYVQYFGGVSALSKQQFLTINGFP
NNYWGWGGEDDDIFNRLVFRGMSISRPNAVVGRCRMIRHSRDKKNEPNP
QRFDRIAHTKETMLSDGLNSLTYQVLDVQRYPLYTQITVDIGTPSPRD
SEQ ID NO: 36
GPASNLTSVPVPHTTALSLPACPEESPLLVGPMLIEFNMPVDLELVAKQ
NPNVKMGGRYAPRDCVSPHKVAIIIPFRNRQEHLKYWLYYLHPVLQRQQ
LDYGIYVINQAGDTIFNRAKLLNVGFQEALKDYDYTCFVFSDVDLIPMN
DHNAYRCFSQPRHISVAMDKFGFSLPYVQYFGGVSALSKQQFLTINGFP
NNYWGWGGEDDDIFNRLVFRGMSISRPNAVVGRCRMIRHSRDKKNEPNP
QRFDRIAHTKETMLSDGLNSLTYQVLDVQRYPLYTQITVDIGTPSPGG

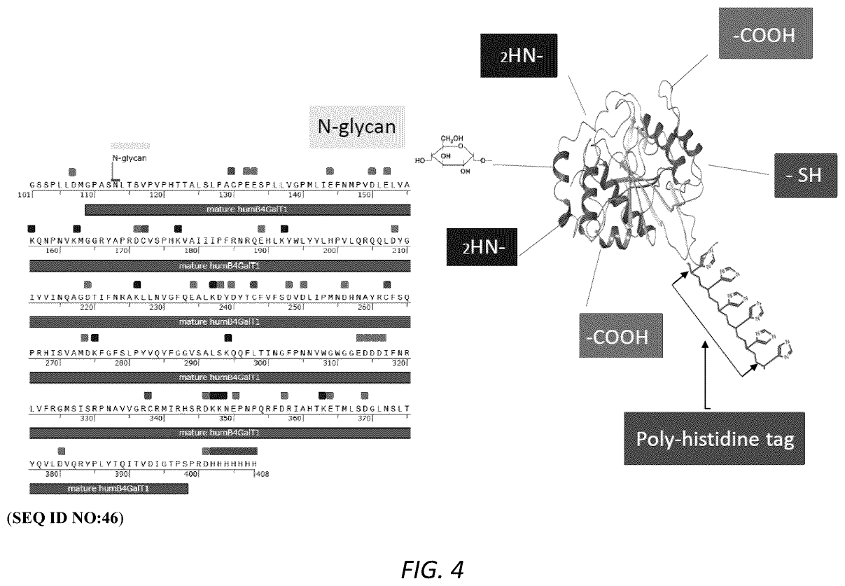

A his-tagged human Beta-1,4-galactosyltransferase 1 (B4GalT) is suitable for use in the methods described herein. A suitable B4GalT can comprise an amino acid sequence that is at least 80% (85%, 90%, 95%, 98% or 100%) identical to SEQ ID NO:37 or SEQ ID NO:38 (a schematic of which is shown in FIG. 4 (amino acids 8-308 of SEQ ID NO: 46)).

SEQ ID NO: 37
MGPASNLTSVPVPHTTALSLPACPEESPLLVGPMLIEFNMPVDLELVAK
QNPNVKMGGRYAPRDCVSPHKVAIIIPFRNRQEHLKYWLYYLHPVLQRQ
QLDYGIYVINQAGDTIFNRAKLLNVGFQEALKDYDYTCFVFSDVDLIPM
NDHNAYRCFSQPRHISVAMDKFGFSLPYVQYFGGVSALSKQQFLTINGF
PNNYWGWGGEDDDIFNRLVFRGMSISRPNAVVGRCRMIRHSRDKKNEPN
PQRFDRIAHTKETMLSDGLNSLTYQVLDVQRYPLYTQITVDIGTPSPGG
HHHHHHHH
SEQ ID NO: 38
MGPASNLTSVPVPHTTALSLPACPEESPLLVGPMLIEFNMPVDLELVAK
QNPNVKMGGRYAPRDCVSPHKVAIIIPFRNRQEHLKYWLYYLHPVLQRQ
QLDYGIYVINQAGDTIFNRAKLLNVGFQEALKDYDYTCFVFSDVDLIPM
NDHNAYRCFSQPRHISVAMDKFGFSLPYVQYFGGVSALSKQQFLTINGF
PNNYWGWGGEDDDIFNRLVFRGMSISRPNAVVGRCRMIRHSRDKKNEPN
PQRFDRIAHTKETMLSDGLNSLTYQVLDVQRYPLYTQITVDIGTPSPRD
HHHHHHH

A visual map of a portion of SEQ ID NO:38 is shown in FIG. 4 (amino acids 8-308 of SEQ ID NO: 46). The two disulfides are marked in the map, as is the N-glycan. The affinity tag is the His-tag at the C-terminus.

A biotin-tagged human Beta-1,4-galactosyltransferase 1 (B4GalT) is suitable for use in the methods described herein. A suitable B4GalT can comprise an amino acid sequence that is at least 80% (85%, 90%, 95%, 98% or 100%) identical to SEQ ID NO:39. In some embodiments, the biotin tag is a variant of biotin.

SEQ ID NO: 39
MGPASNLTSVPVPHTTALSLPACPEESPLLVGPMLIEFNMPVDLELVAK
QNPNVKMGGRYAPRDCVSPHKVAIIIPFRNRQEHLKYWLYYLHPVLQRQ
QLDYGIYVINQAGDTIFNRAKLLNVGFQEALKDYDYTCFVFSDVDLIPM
NDHNAYRCFSQPRHISVAMDKFGFSLPYVQYFGGVSALSKQQFLTINGF
PNNYWGWGGEDDDIFNRLVFRGMSISRPNAVVGRCRMIRHSRDKKNEPN
PQRFDRIAHTKETMLSDGLNSLTYQVLDVQRYPLYTQITVDIGTPSPG
G-Biotin

### Supports

In some embodiments, the support, e.g., solid support, e.g., porous solid support, is a resin, column, array, microarray, solid phase.

In some embodiments, the support material can comprise a membrane, a bead, a gel, a cassette, a column, a chip, a slide, a plate, an array, a microarray, or a monolith. In some embodiments, the support material may comprise a hydrophilic compound, a hydrophobic compound, an oleophobic compound, an oleophilic compound, or any combination thereof. In some embodiments, the support material may comprise a polymer or a copolymer.

Examples of suitable support materials, include, but are not limited to polyether sulfone, polyamide, e.g., agarose, cellulose, a polysaccharide, polytetrafluoroethylene, polysulfone, polyester, polyvinylidene fluoride, polypropylene, a fluorocarbon, e.g. poly (tetrafluoroethylene-co-perfluoro(alkyl vinyl ether)), poly carbonate, polyethylene, glass, polycarbonate, polyacrylate, polyacrylamide, poly(azolactone), polystyrene, ceramic, nylon and metal.

In some embodiments, the support comprises a metal (e.g. metal chelate), Nickel (e.g. Ni2+), Cobalt (e.g. Co2+), chitin, maltose, GSH, an antibody or nanobody, a FLAG-binding antibody or nanobody, a HA-binding antibody or nanobody, a Myc-binding antibody or nanobody, an NE-binding antibody or nanobody, streptavidin, biotin, calmodulin, a Spot-binding antibody or nanobody, variants thereof, and combinations thereof.

In some embodiments, the support comprises a ligand that binds an affinity tag, e.g., an affinity tag of a polypeptide comprising a B4GalT, e.g., a poly-histidine tag, as described herein. In some embodiments, the support comprises a ligand selected from the group consisting of nickel (e.g., Ni-NTA or Ni-IDA), cobalt, and combinations thereof.

In some embodiments, the support is a bead, e.g., a magnetic bead. In some embodiments, the support is a magnetic agarose bead. In some embodiments, the magnetic agarose bead is a magnetic sepharose bead. In some embodiments, the support is a resin. In some embodiments, the support is an agarose resin. In some embodiments, the agarose resin is a sepharose resin.

In some embodiments, the magnetic agarose bead or agarose resin comprises an agarose gel of about 1% to about 10% w/v. In some embodiments, the magnetic agarose bead or agarose resin comprises an agarose gel of about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% w/v.

In some embodiments, the pore size range of the support is from about 20 to about 130 nm. In some embodiments, the pore size range of the support is about 20 to about 120, about 20 to about 110, about 20 to about 100, about 20 to about 90, about 20 to about 80, about 20 to about 70, about 20 to about 60, about 20 to about 50, about 20 to about 40, about 20 to about 30, about 30 to about 130, about 30 to about 120, about 30 to about 110, about 30 to about 100, about 30 to about 90, about 30 to about 80, about 30 to about 70, about 30 to about 60, about 30 to about 50, about 30 to about 40, about 40 to about 130, about 40 to about 120, about 40 to about 110, about 40 to about 100, about 40 to about 90, about 40 to about 80, about 40 to about 70, about 40 to about 60, about 40 to about 50, about 50 to about 130, about 50 to about 120, about 50 to about 110, about 50 to about 100, about 50 to about 90, about 50 to about 80, about 50 to about 70, about 50 to about 60, about 60 to about 130, about 60 to about 120, about 60 to about 110, about 60 to about 100, about 60 to about 90, about 60 to about 80, about 60 to about 70, about 70 to about 130, about 70 to about 120, about 70 to about 110, about 70 to about 100, about 70 to about 90, about 70 to about 80, about 80 to about 130, about 80 to about 120, about 80 to about 110, about 80 to about 100, about 80 to about 90, about 90 to about 130, about 90 to about 120, about 90 to about 110, about 90 to about 100, about 100 to about 130, about 100 to about 120, about 100 to about 110, about 110 to about 130, about 110 to about 120, or about 120 to about 130 nm.

In some embodiments, the support, e.g., bead or resin, e.g., magnetic bead or magnetic resin, is from about 10 to about 350 μm in size, e.g., in diameter. In some embodiments, the support, e.g., bead, e.g., magnetic bead is from about 10 to about 170, about 10 to about 160, about 10 to about 150, about 10 to about 140, about 10 to about 130, about 10 to about 120, about 10 to about 110, about 10 to about 100, about 10 to about 90, about 10 to about 80, about 10 to about 70, about 10 to about 60, about 10 to about 50, about 10 to about 40, about 10 to about 30, about 10 to about 20, about 20 to about 170, about 20 to about 160, about 20 to about 150, about 20 to about 140, about 20 to about 130, about 20 to about 120, about 20 to about 110, about 20 to about 100, about 20 to about 90, about 20 to about 80, about 20 to about 70, about 20 to about 60, about 20 to about 50, about 20 to about 40, about 20 to about 30, about 30 to about 170, about 30 to about 160, about 30 to about 150, about 30 to about 140, about 30 to about 130, about 30 to about 120, about 30 to about 110, about 30 to about 100, about 30 to about 90, about 30 to about 80, about 30 to about 70, about 30 to about 60, about 30 to about 50, about 30 to about 40, about 40 to about 170, about 40 to about 160, about 40 to about 150, about 40 to about 140, about 40 to about 130, about 40 to about 120, about 40 to about 110, about 40 to about 100, about 40 to about 90, about 40 to about 80, about 40 to about 70, about 40 to about 60, about 40 to about 50, about 50 to about 170, about 50 to about 160, about 50 to about 150, about 50 to about 140, about 50 to about 130, about 50 to about 120, about 50 to about 110, about 50 to about 100, about 50 to about 90, about 50 to about 80, about 50 to about 70, about 50 to about 60, about 60 to about 170, about 60 to about 160, about 60 to about 150, about 60 to about 140, about 60 to about 130, about 60 to about 120, about 60 to about 110, about 60 to about 100, about 60 to about 90, about 60 to about 80, about 60 to about 70, about 70 to about 170, about 70 to about 160, about 70 to about 150, about 70 to about 140, about 70 to about 130, about 70 to about 120, about 70 to about 110, about 70 to about 100, about 70 to about 90, about 70 to about 80, about 80 to about 170, about 80 to about 160, about 80 to about 150, about 80 to about 140, about 80 to about 130, about 80 to about 120, about 80 to about 110, about 80 to about 100, about 80 to about 90, about 90 to about 170, about 90 to about 160, about 90 to about 150, about 90 to about 140, about 90 to about 130, about 90 to about 120, about 90 to about 110, about 90 to about 100, about 100 to about 170, about 100 to about 160, about 100 to about 150, about 100 to about 140, about 100 to about 130, about 100 to about 120, about 100 to about 110, about 110 to about 170, about 110 to about 160, about 110 to about 150, about 110 to about 140, about 110 to about 130, about 110 to about 120, about 120 to about 170, about 120 to about 160, about 120 to about 150, about 120 to about 140, about 120 to about 130, about 130 to about 170, about 130 to about 160, about 130 to about 150 about 130 to about 140, about 140 to about 170, about 140 to about 160, about 140 to about 150, about 150 to about 170, about 150 to about 160, or about 160 to about 170 μm in size, e.g., in diameter. In some embodiments, the support is about 10 to about 40 μm in diameter. In some embodiments, the support is about 10 μm in diameter.

Methods of Hypersialylation with Immobilized B4-GalT

In some embodiments, described herein, inter alia, is a method of preparing hypersialylated (hsIgG), the method comprising: (a) providing a mixture of IgG antibodies, (b) incubating the mixture of IgG antibodies in a reaction mixture comprising β1,4-Galactosyltransferase I (β4GalT1, also called B4GalT) bound to a solid support and UDP-Gal to produce galactosylated IgG antibodies; (c) incubating the galactosylated IgG antibodies in a reaction mixture comprising ST6Gal1 (also called ST6) and CMP-NANA, thereby creating the hsIgG preparation.

Suitable β4GalT1, e.g., human B4GalT, e.g., human B4Galt1, as well as orthologs, mutants, and variants thereof, including enzymatically active portions of beta-1,4-galactosyltransferase (B4GalT), e.g., human B4GalT, e.g., human B4Galt1, as well as orthologs, mutants, and variants thereof, along with fusion proteins and polypeptides comprising the same, are described herein.

Suitable ST6Gal1, e.g., human ST6Gal1, as well as orthologs, mutants, and variants thereof, including enzymatically active portions of ST6Gal1, e.g., human ST6Gal1, as well as orthologs, mutants, and variants thereof, along with fusion proteins and polypeptides comprising the same, are described herein.

In some embodiments, the β4GalT1 is bound to the solid support through at least one affinity tag. Suitable affinity tags and solid supports are described herein.

In some embodiments, the β1,4-Galactosyltransferase I (β4GalT1) bound to a solid support (e.g. resin, column, array, etc.) is separated from the galactosylated IgG antibodies prior to step (b).

In some embodiments, GMP-NANA is added 1, 2, 3, or more times during the sialylation reaction.

In some embodiments, the IgG antibodies comprise IgG antibodies isolated from at least 1000 donors. In some embodiments, at least 70% w/w of the IgG antibodies are IgG1 antibodies. In some embodiments, at least 90% of the donor subjects have been exposed to a virus. In some embodiments, about 60%, 65%, 70%, 75%, 80%, or 85% of the branched glycans on the IgG antibodies in the hsIgG preparation have a sialic acid on both the α1,3 branch and the α1,6 branch. In some embodiments, at least 60%, 65%, 70%, 75%, 80%, or 85% of the branched glycans on the Fab domain of the IgG antibodies in the hsIgG preparation have a sialic acid on both the α 1,3 arm and the α 1,6 arm that is connected through a NeuAc-α 2,6-Gal terminal linkage; and at least 60%, 65%, 70%, 75%, 80%, or 85% of the branched glycans on the Fc domain of the IgG antibodies in the hsIgG preparation have a sialic acid on both the α 1,3 arm and the α 1,6 arm that is connected through a NeuAc-α 2,6-Gal terminal linkage.

### EXAMPLES

### Example 1: Hypersialylated IgG Preparation

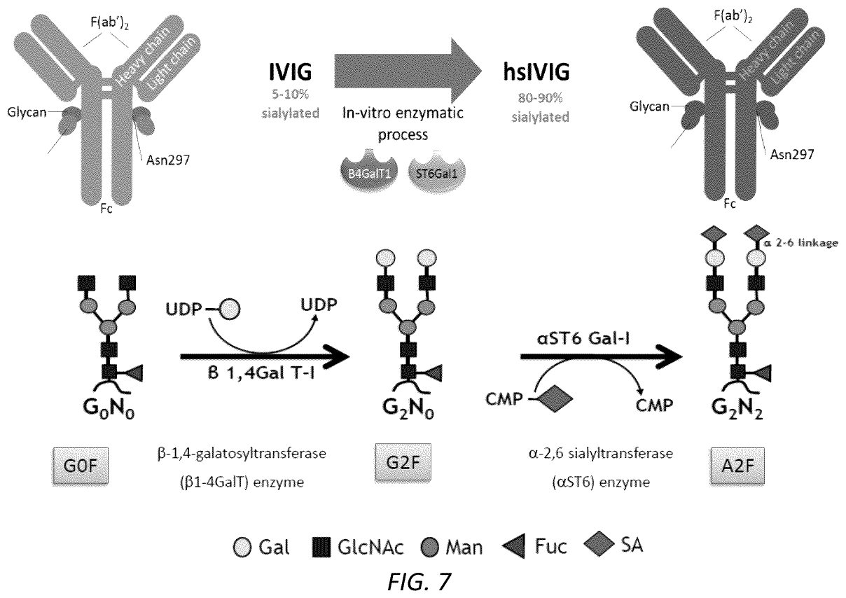

IgG in which more than 60% of the overall branched glycans are disialylated can be prepared as follows. An exemplary reaction is shown in FIG. 7.

Briefly, a mixture of IgG antibodies was exposed to a sequential enzymatic reaction using β1,4 galactosyltransferase 1 (B4-GalT or β4GalT1) and α2,6-sialyltransferase (ST6-Gal1) enzymes. The B4-GalT does not need to be removed from the reaction before addition of ST6-Gal1 and no partial or complete purification of the product is needed between the enzymatic reactions. However, multiple purifications steps to remove the enzymes from the hsIgG product typically follow sialylation.

The galactosyltransferase enzyme selectively adds galactose residues to pre-existing asparagine-linked glycans. The resulting galactosylated glycans serve as substrates to the sialic acid transferase enzyme which selectively adds sialic acid residues to cap the asparagine-linked glycan structures attached to. Thus, the overall sialylation reaction employed two sugar nucleotides (uridine 5′-diphosphogalactose (UDPGal) and cytidine-5′-monophospho-N-acetylneuraminic acid (CMP-NANA)). The latter is replenished periodically to increase disialylated product relative to monosialylated product. The reaction includes the co-factor manganese chloride.

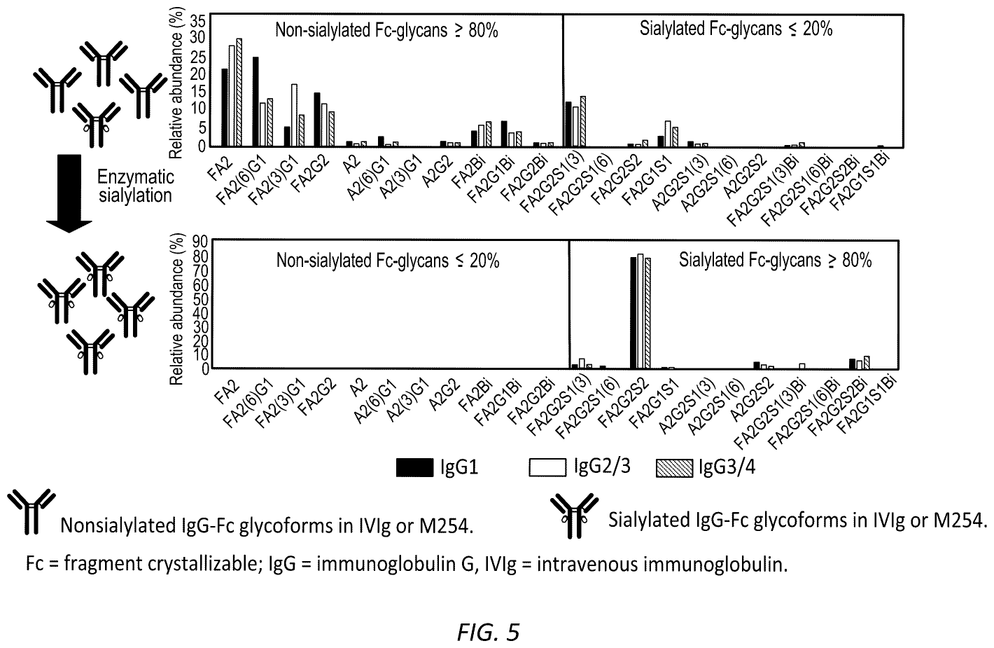

A representative example of the IgG-Fc glycan profile for such a reaction starting with IVIg and the reaction product is shown in FIG. 5. In FIG. 5, the left panel is a schematic representation of enzymatic sialylation reaction to transform IgG to hsIgG; the right panel is the IgG Fc glycan profile for the starting IVIg and hsIgG. In this study, glycan profiles for the different IgG subclasses are derived via glycopeptide mass spectrometry analysis. The peptide sequences used to quantify glycopeptides for different IgG subclasses were: IgG1=EEQYNSTYR (SEQ ID NO:40), IgG2/3 EEQFNSTFR (SEQ ID NO:41), IgG3/4 EEQYNSTFR (SEQ ID NO:42) and EEQFNSTYR (SEQ ID NO:43).

The glycan data is shown per IgG subclass. Glycans from IgG3 and IgG4 subclasses cannot be quantified separately. As shown, for IVIg the sum of all the nonsialylated glycans is more than 80% and the sum of all sialylated glycans is <20%. For the reaction product, the sum for all nonsialylated glycans is <20% and the sum for all sialylated glycans is more than 80%. Nomenclature for different glycans listed in the glycoprofile use the Oxford notation for N linked glycans.

### Example 2: Hypersialylated IgG Preparation with Immobilized B4-GalT

IgG in which more than 60% of the overall branched glycans are disialylated can be prepared as follows.

Briefly, a mixture of IgG antibodies was exposed to a sequential enzymatic reaction using His-tagged β1,4 galactosyltransferase 1 (B4-GalT or β4GalT1) and α2,6-sialyltransferase (ST6-Gal1) enzymes. The B4-GalT was immobilized on a nickel Sepharose resin. No partial or complete purification of the product is needed before the ST6-Gal1 enzymatic reactions.

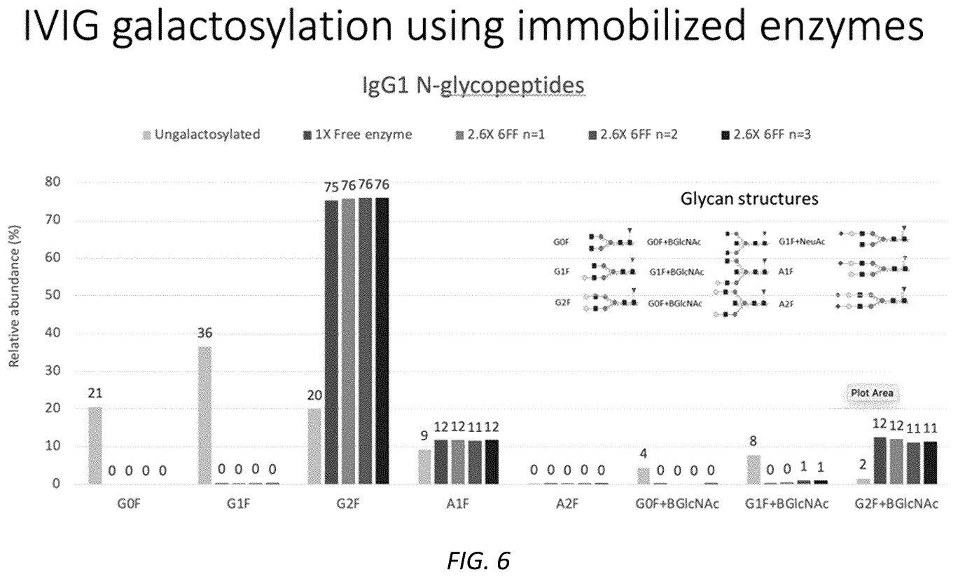

Coupling of the His-β4GalT1 to the nickel Sepharose resin occurred by interaction of the chelated nickel molecules by the 8× poly-histidine tag (SEQ ID NO: 30) at the C-terminal end of the β4GalT1. Immobilization was optimized in an aqueous solution suitable for the stability of the enzyme and was shown to be stable for greater than 21 days at 3TC. This stability means that minimal leaching of the enzyme (and therefore Ni) occurred over this time period and that multiple batches of product could be generated from one lot of immobilized enzyme. The amount of immobilized enzyme needed for galactosylation of IVIg was determined by performing protein and enzyme activity assays and quantifying the specific activity, which was 50% to 80% of the soluble enzyme. Galactosylation of IVIg occurred over 72 hours at 37° C. in MOPS buffer at pH 7.4 with UDP-Gal. Constant mixing was carried out using a tube rotator. The immobilized enzyme was filtered away and the extent of galactosylation was characterized and quantified by mass spec methods. The extent of galactosylation was found to be equivalent to that seen for the soluble enzyme and was nearly completely G2F for IgG1, 2, 3, and 4. IgG1 results of 3 separate reactions are shown in FIG. 6, which is bar graph showing relative abundance of the N-glycopeptides following galactosylation. Free/soluble enzyme (1×) is in column 2 in each group, and three different experiments of immobilizing the B4-GalT are in columns 3, 4, and 5 in each group. The starting Immunoglobins are in column 1 in each group.

Thus, about 50%-80% less enzyme used in the galactosylation reaction resulted in similar or more proper glycan structure than soluble enzyme.

### Example 2: Immobilization of B4-GalT

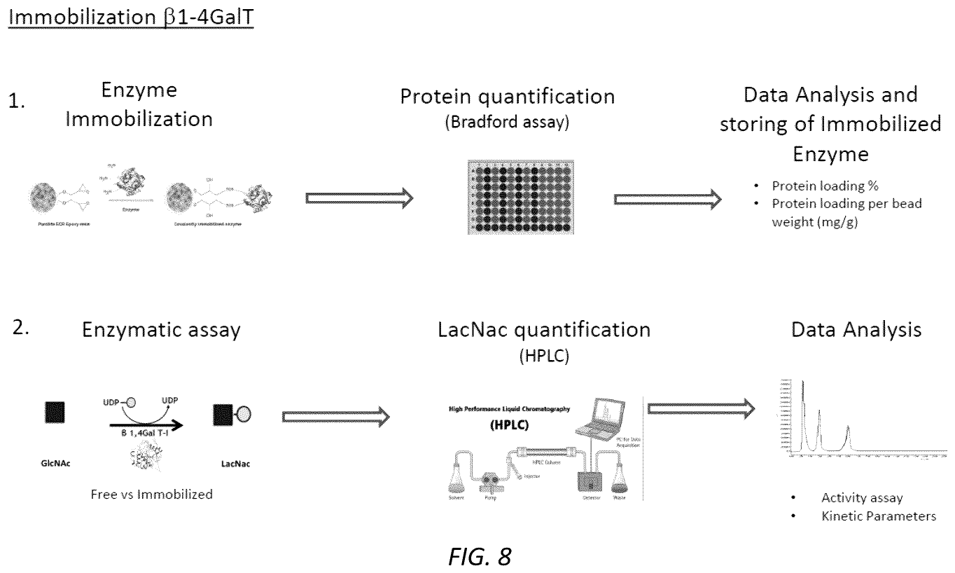

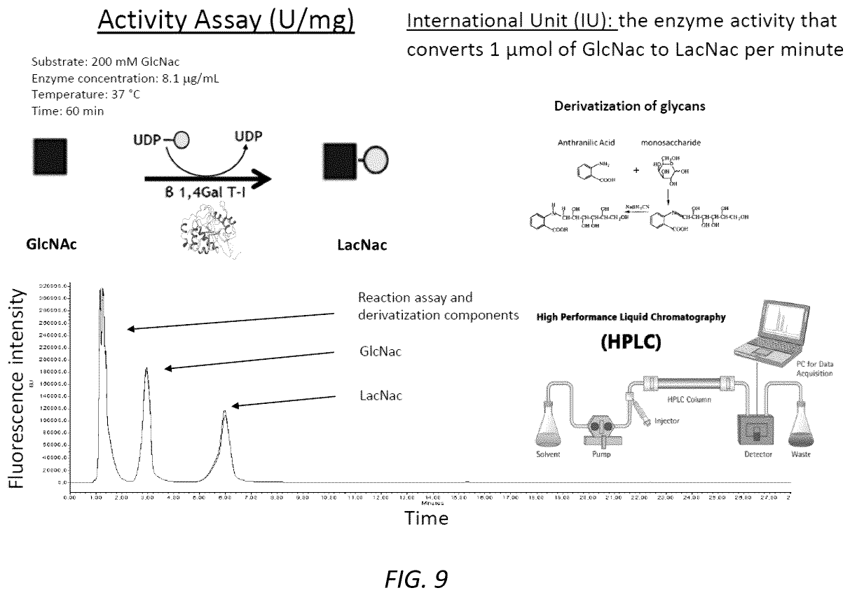

An enzymatically active portion of B4-GalT (SEQ ID NO:38, FIG. 4) was immobilized using a variety of techniques described herein and analyzed as shown in FIGS. 8-9.

Adsorption with Nickel Loaded Beads Achieved Up to 50% Enzyme Activity

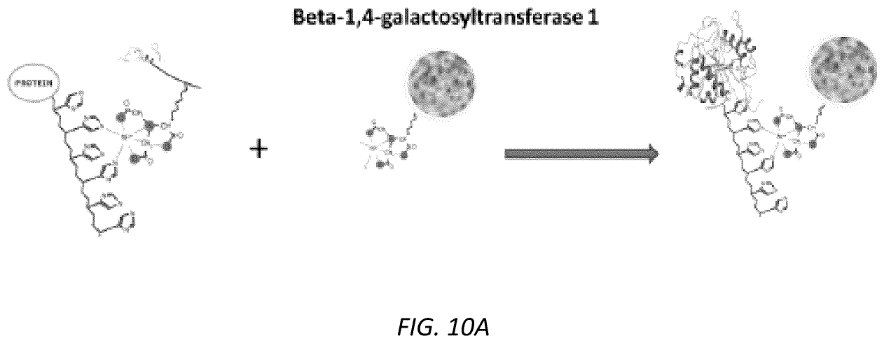

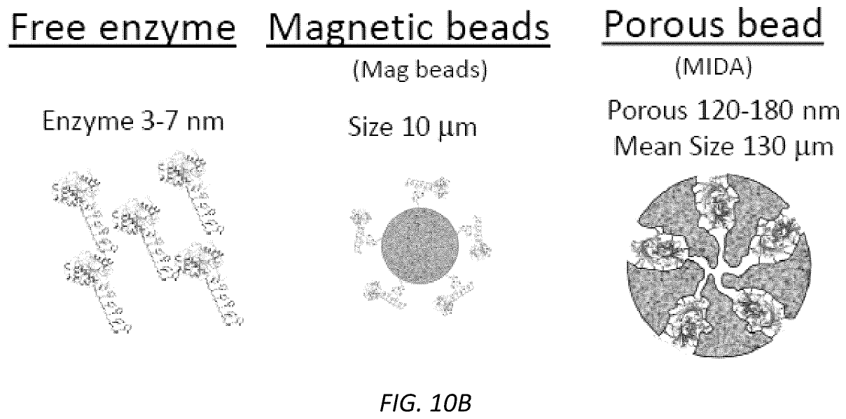

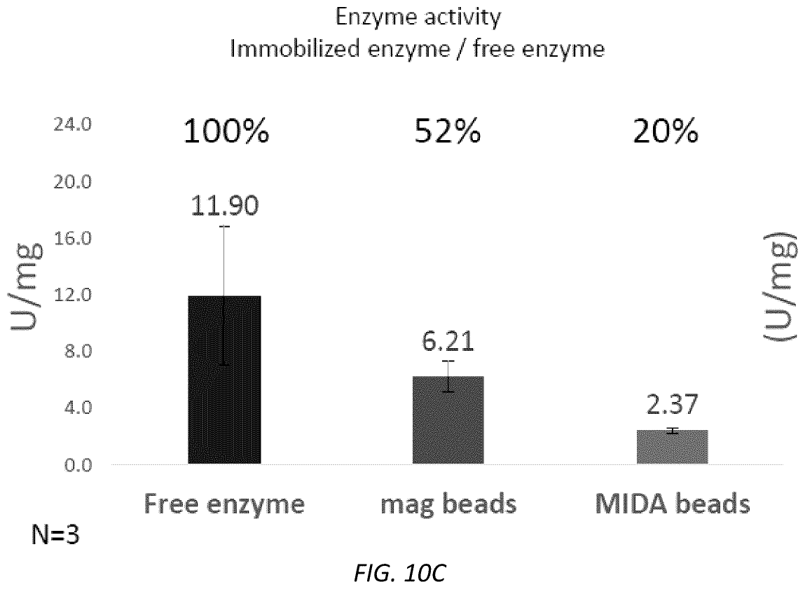

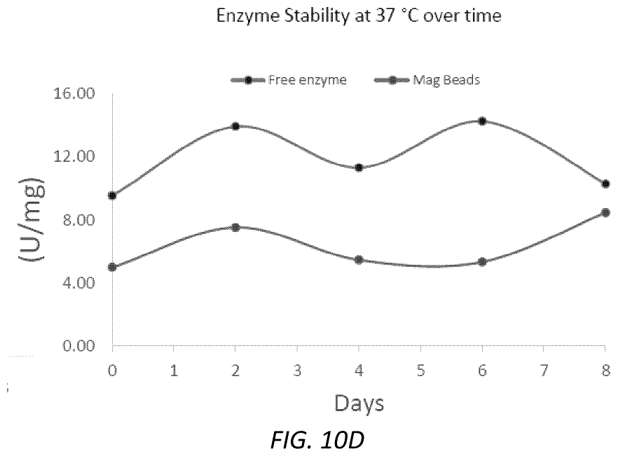

As shown in FIGS. 10A-10D, the poly-histidine tag of B4-GalT was used as an attachment point for enzyme immobilization (FIG. 10A) to either magnetic beads or porous beads (FIG. 10B). B4-GalT immobilized on 10 μm magnetic beads achieved ˜52% of the activity of free enzyme, whereas B4-GalT immobilized on porous (120-180 nm) porous beads with a mean size of 130 μm achieved ˜20% of the activity of free enzyme (FIG. 10C); N=3. Stability at 37° C. over time is shown in FIG. 10D.

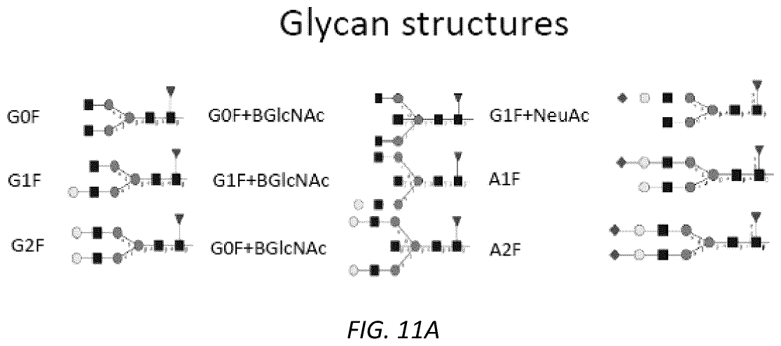

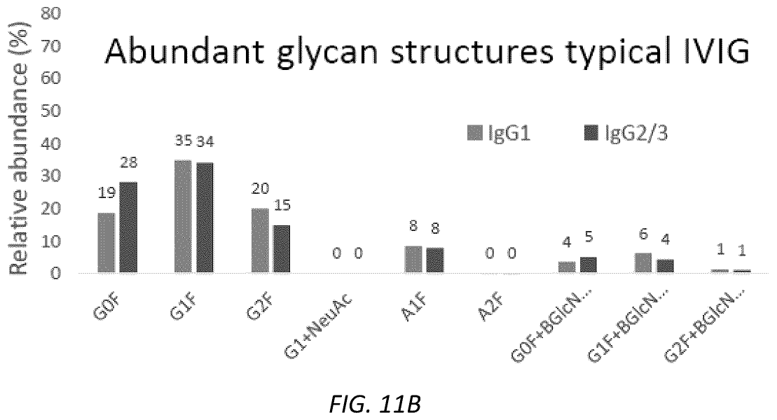

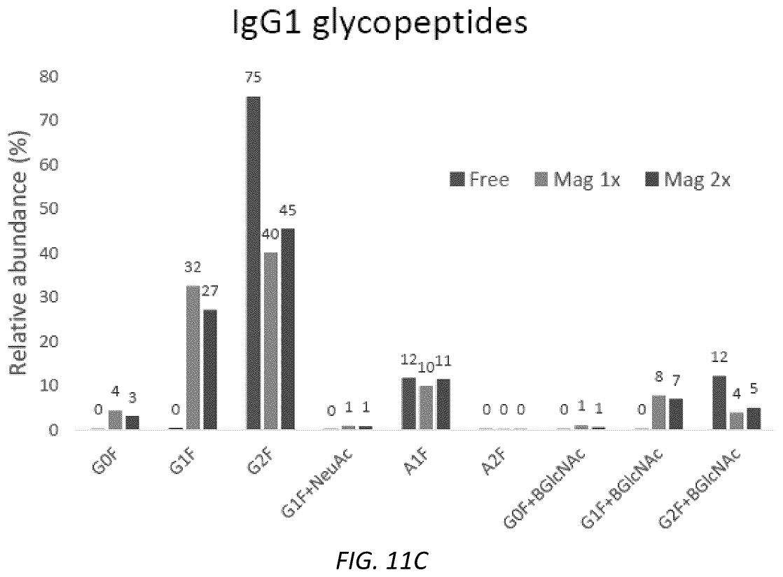

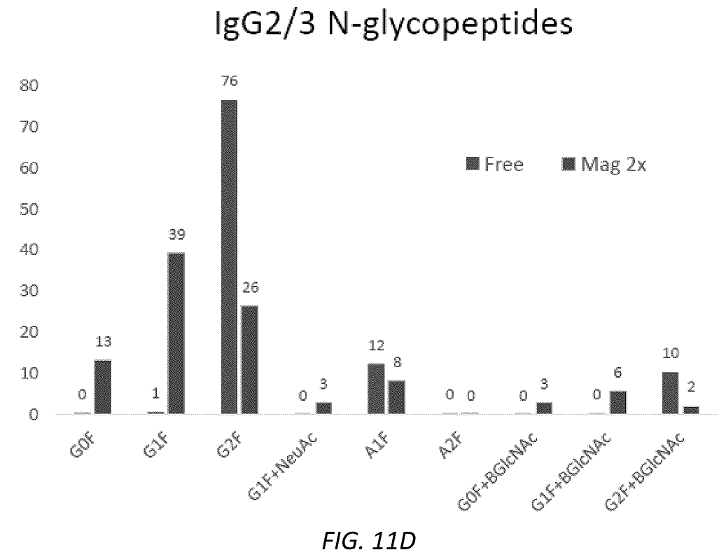

As shown in FIGS. 11A-11D, the immobilized enzymes were able to galactosylate IVIGS. FIG. 11A shows various glycan structures. FIG. 11B shows abundant glycan structures typical to IVIG. FIG. 11C shows relative abundance of IgG1 glycopeptides after galactosylation with magnetic bead immobilized B4-GalT. FIG. 11D shows relative abundance of IgG2/G3 N-glycopeptides after galactosylation with magnetic bead immobilized B4-GalT. 1×=same number of units as free enzyme reaction.

### Amine Coupling Achieved Up to 17% Enzyme Activity

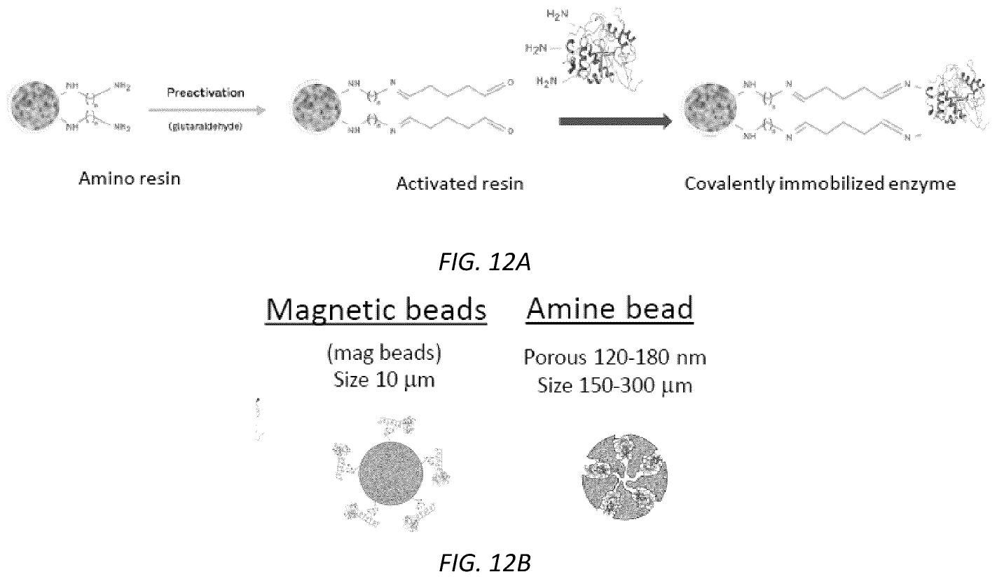

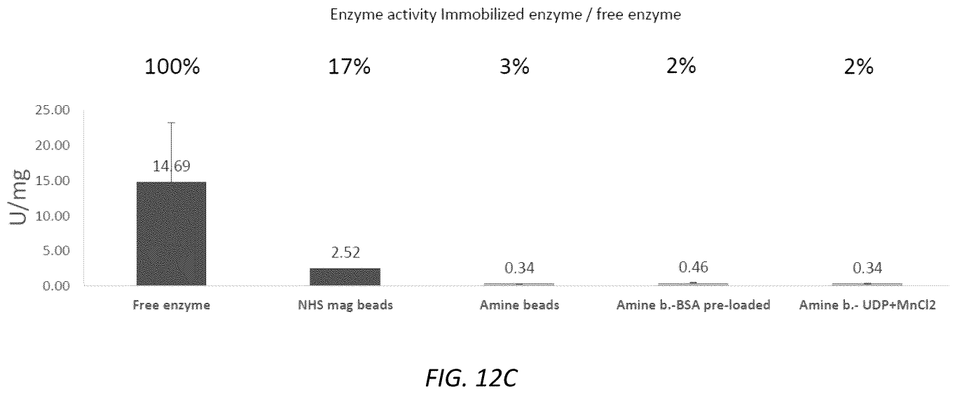

As shown in FIGS. 12A-12C, 4-GalT was immobilized via amine coupling chemistry (FIG. 12A) to either magnetic beads or porous beads (FIG. 12B). B4-GalT immobilized on 10 μm NHS magnetic beads achieved ˜17% of the activity of free enzyme, whereas B4-GalT immobilized on porous (120-180 nm) amine beads size 150-300 μm achieved ˜2-3% of the activity of free enzyme (FIG. 10C); N=3. Stability at 37° C. over time is shown in FIG. 10D.

### Epoxy Coupling Reduced 99% Enzyme Activity

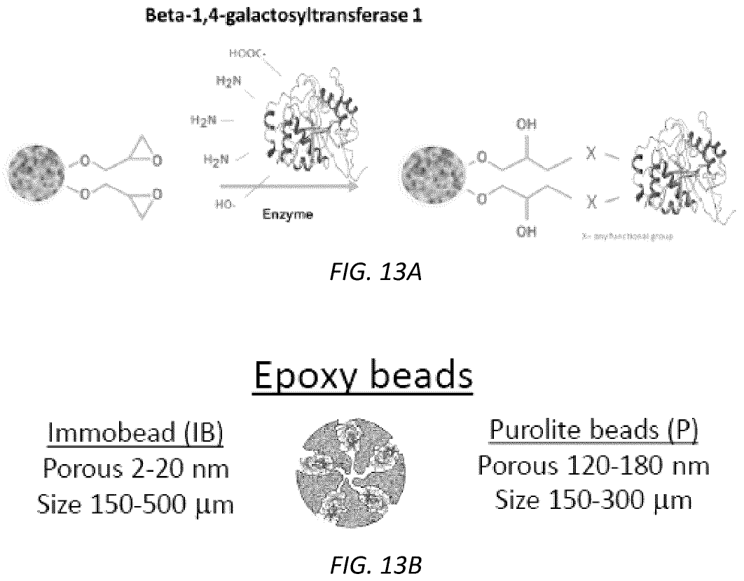

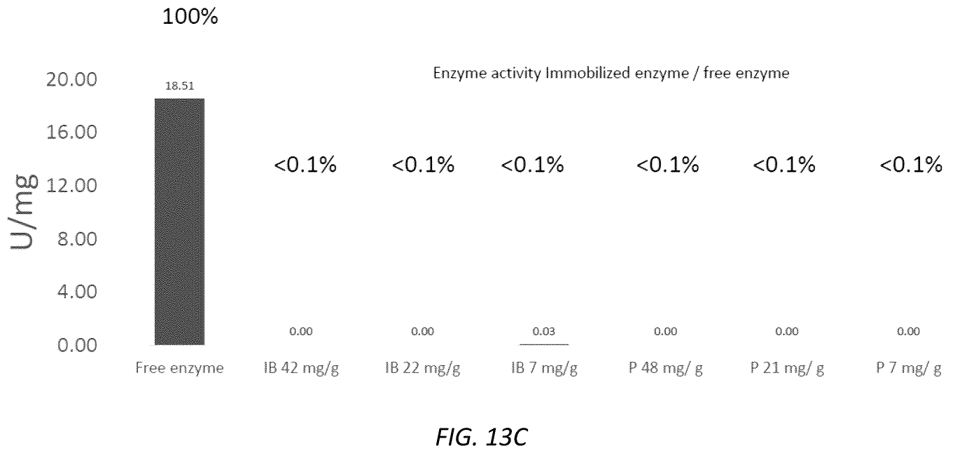

As shown in FIGS. 13A-13C, 4-GalT was immobilized via multi-point epoxy chemistry (FIG. 12A) to either Immobead (IB) or Purolite (P) porous beads, with porosity of 2-23 nm and 120-180 nm, respectively, and size of 150-500 μm-150-300 μm, respectively (FIG. 12B). Activity of the immobilized enzyme was less than ˜0.1% (FIG. 12C).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of galatosylating IgG antibodies, the method comprising:
(a) providing a mixture of IgG antibodies; and
(b) incubating the mixture of IgG antibodies in a reaction mixture comprising:
a polypeptide comprising an enzymatically active portion of human β1,4-Galactosyltransferase I (β4GalT1) bound to a solid support; and
UDP-Gal, thereby producing galactosylated IgG antibodies.

2. A method of preparing hypersialylated (hsIgG), the method comprising:
(a) providing galactosylated IgG antibodies produced by the method of claim 1; and
(b) incubating the galactosylated IgG antibodies in a reaction mixture comprising:
a polypeptide comprising human ST6Gal1 or enzymatically active portion thereof; and
CMP-NANA, thereby producing hsIgG.

3. The method of claim 2, further comprising:
(c) isolating the polypeptide comprising an enzymatically active portion of human β1,4-Galactosyltransferase I (β4GalT1) bound to a solid support from the reaction mixture, thereby producing recycled β4GalT1; and
repeating steps (a)-(b), wherein the β4GalT1 in the reaction mixture is the β4GalT1 isolated in step (c).

4. A method of preparing hypersialylated (hsIgG), the method comprising
(a) providing a mixture of IgG antibodies,
(b) incubating the mixture of IgG antibodies in a reaction mixture comprising:
a polypeptide comprising an enzymatically active portion of human β1,4-Galactosyltransferase I (β4GalT1) bound to a solid support; and
UDP-Gal, thereby producing galactosylated IgG antibodies; and
(c) incubating the galactosylated IgG antibodies in a reaction mixture comprising:
a polypeptide comprising human ST6Gal1 or enzymatically active portion thereof; and
CMP-NANA, thereby producing hsIgG.

5. The method of claim 4, further comprising:
(d) isolating the polypeptide comprising an enzymatically active portion of human β1,4-Galactosyltransferase I (β4GalT1) bound to a solid support from the reaction mixture, thereby producing recycled β4GalT1; and
repeating steps (a)-(c), wherein the β4GalT1 in the reaction mixture is the β4GalT1 isolated in step (d).

6. The method of claim 1, wherein the human β1,4-Galactosyltransferase I (β4GalT1) bound to a solid support is separated from the galactosylated IgG antibodies after step (b).

7. The method of claim 1, wherein the enzymatically active portion of human β4GalT1 comprises SEQ ID NO:8.

8. The method of claim 7, wherein the polypeptide comprising the enzymatically active portion of human β4GalT1 is at least 85% identical SEQ ID NO: 37, 38, or 39, or a variant thereof having 1, 2, 3, 4, 5, 6, 7, 8, 9, or 10 amino acid substitutions, additions, or subtractions.

9. The method of claim 2, wherein the human ST6Gal1 or enzymatically active portion thereof comprises SEQ ID NO:14.

10. The method of claim 1, wherein the polypeptide comprising an enzymatically active portion of human β4GalT1 further comprises an affinity tag, wherein the affinity tag is attached to the solid support.

11. The method of one of claim 10, wherein the affinity tag is C-terminal.

12. The method of claim 10, wherein the at least one tag is selected from the group comprising polyhistidine, chitin binding protein (CBP), glutathione S-transferase (GST), maltose-binding protein (MBP), hemagglutinin (HA), Myc, streptavidin-binding peptide (SBP), calmodulin-tag, Spot-tag, a streptavidin tag, FLAG-tag, biotin, and combinations thereof.

13. The method of claim 12, wherein the polyhistidine tag comprises 3, 4, 5, 6, 7, 8, 9, 10, 11, 12, 13, 14, 15, 16, 17, 18, 19, or 20 histidines.

14. The method of claim 13, wherein the polyhistidine tag comprises 7 or 8 histidines.

15. The method of claim 1, wherein the solid support is a magnetic bead.

16. The method of claim 1, wherein the IgG antibodies comprise IgG antibodies isolated from at least 1000 donors.

17. The method of claim 1, wherein at least 70% w/w of the IgG antibodies are IgG1 antibodies.

18. The method of claim 1, wherein at least 90% of the donor subjects have been exposed to a virus.

19. The method of claim 2, wherein about 60%, 65%, 70%, 75%, 80%, or 85% of the branched glycans on the IgG antibodies in the hsIgG preparation have a sialic acid on both the α1,3 branch and the α1,6 branch.

20. The method of claim 1, wherein at least 60%, 65%, 70%, 75%, 80%, or 85% of the branched glycans on the Fab domain of the IgG antibodies in the hsIgG preparation have a sialic acid on both the α 1,3 arm and the α 1,6 arm that is connected through a NeuAc-α 2,6-Gal terminal linkage; and at least 60%, 65%, 70%, 75%, 80%, or 85% of the branched glycans on the Fc domain of the IgG antibodies in the hsIgG preparation have a sialic acid on both the α 1,3 arm and the α 1,6 arm that is connected through a NeuAc-α 2,6-Gal terminal linkage.

21. A polypeptide comprising:
an enzymatically active portion of human β1,4-Galactosyltransferase I (β4GalT1); and
an affinity tag,
wherein the polypeptide is bound to a solid support.

22. The polypeptide of claim 12, wherein the enzymatically active portion of β4GalT1 comprises SEQ ID NO:8.

23. The polypeptide of claim 21, wherein the affinity tag comprises a poly-histidine tag selected from the group consisting of HHHH (SEQ ID NO:26), HHHHH (SEQ ID NO:27), HHHHHH, (SEQ ID NO:28), HHHHHHH (SEQ ID NO:29), HHHHHHHH (SEQ ID NO:30), HHHHHHHHH (SEQ ID NO:31), and HHHHHHHHHH (SEQ ID NO:32).

24. The polypeptide of claim 21, wherein the solid support is an agarose magnetic bead.

25. A composition comprising:
the polypeptide of claim 21;
a ST6Gal1;
UDP-Gal;
CMP-NANA; and
IgG antibodies.
